(19) United States
(12) Patent Application Publication
(10) Pub. No.: US 2026/0365637 A1
(43) Pub. Date: Nov. 19, 2026

(54) DUAL EZH2-HSP90 INHIBITORS

(71) Applicant: TAIPEI MEDICAL UNIVERSITY, Taipei City (TW)

(72) Inventors: Jing-Ping LIOU, Taipei City (TW); Tsung-I HSU, Taipei City (TW)

(21) Appl. No.: 19/483,700

(22) PCT Filed: May 10, 2024
(86) PCT No.: PCT/US2024/028914

§ 371 Date: Nov. 12, 2025

Related U.S. Application Data
(60) Provisional application No. 63/501,939, filed on May 12, 2023.

(51) Int. Cl. C07D 405/12; A61K 31/4433; A61P 35/04

(52) U.S. Cl. CPC C07D 405/12; A61K 31/4433; A61P 35/04

(57) ABSTRACT

The present disclosure relates to first-in-class dual EZH2-HSP90 inhibitors, process of preparing the same and uses thereof in treatment of diseases/disorders mediated by EZH2. HSP90 or both, in particular glioblastoma.

### PRIORITY INFORMATION

This application is a 371 National Phase of International Patent Application No. PCT/US24/28914, filed May 10, 2024, which claims benefit of and priority to U.S. Provisional Patent Application No. 63/501,939, filed May 12, 2023, entitled, “FIRST-IN-CLASS DUAL EZH2-HSP90 INHIBITORS,” the contents of which are incorporated by reference herewith in their entirety.

### FILED OF THE INVENTION

The present disclosure relates to EZH2-HSP90 inhibitors, the process of preparing the same and uses thereof in treatment of diseases/disorders mediated by EZH2, HSP90 and/or both, in particular glioblastoma.

### BACKGROUND OF THE INVENTION

Glioblastoma (GBM), also known as grade IV astrocytoma, is characterized by a genetically unstable and highly infiltrative population of cells with a high degree or capability of invasion. GBM is considered to be the most devastating intracranial cancer and is of two types, primary or de novo GBM (most common and aggressive form) and secondary GBM (rare and less aggressive). GBM tumor primarily originates from abnormal astrocytic cells, predominantly found in the frontal lobe, and can also metastasize to other parts of the brain via the ventricular system or corpus callosum, with sporadic incidents of spreading to the spinal cord.

Surgical debulking flanked by radiotherapy and chemotherapy represents the currently recommended multimodal approach for the treatment of GBM; however, this trimodal therapy has been unable to attain the desired survival benefits.

Noteworthy to mention that only a few available anti-GBM chemotherapeutic options have also marred GBM treatment therapy. Hindering factors those are pinpointed to hurdle the clinical success of the currently recommended chemotherapeutic regimens are methylguanine DNA methyltransferase mediated innate resistance to temozolomide (oral alkylating agent, only first-line agent for GBM), cytological heterogeneity of GBM and existence of a highly tumorigenic population of cells (glioma stem cells, GSCs) (Safari, M; Khoshnevisan, A. Cancer stem cells and chemoresistance in glioblastoma multiform: a review article. J. Stem Cells 2015, 10 (4), 271; Zhang, F.; Xu, C.-L.; Liu, C.-M Drug delivery strategies to enhance the permeability of the blood-brain barrier for treatment of glioma. Drug Des. Devel. Ther. 2015, 9, 2089).

### SUMMARY OF THE INVENTION

The present disclosure provides dual EZH2-HSP90 inhibitors for preventing and/or treating diseases/disorders mediated by EZH2, HSP90 and/or both (such as glioblastoma).

In one embodiment, the present disclosure provides a compound of Formula (I),

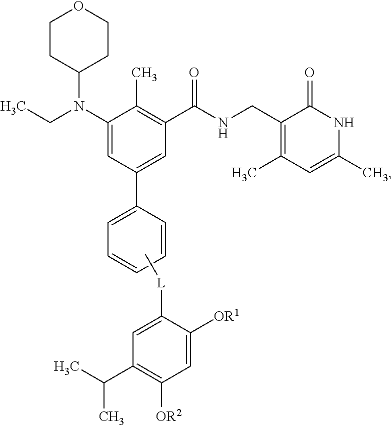

or a pharmaceutically acceptable salt, hydrate, solvate, prodrug or isomer thereof,
wherein L is a linker selected from the group consisting of -A-NR—C(═Y)—B— and -A-C(═Y)—NR—B—;
A and B are independently selected from the group consisting of a direct bond, C₁-C₃ alkyl and C₁-C₃ alkoxy;
R is selected from the group consisting of H, linear or branched C₁-C₆ alkyl, linear or branched C₁-C₆ alkoxy, C₆-C₁₀ aryl and C₅-C₁₀ heteroaryl;
Y is selected from the group consisting of O, S and N; and
R¹ and R² are independently selected from H, halo, C₁-C₆ alkyl and C₁₆-C₂₀ (poly)unsaturated fatty acids;
wherein the alkyl and alkoxyl can be optionally substituted by halogen, hydroxy, amino or C₆-C₁₀ aryl or C₅-C₁₀ heteroaryl; and
wherein the aryl and heteroaryl can be optionally substituted by halogen, hydroxy, amino, C₁-C₃ alkyl or C₁-C₃ alkoxy.

In some embodiments, L is -A-NR—C(═Y)—B— and -A-C(═Y)—NR—B—, wherein Y is O, S or N; A and B are independently selected from the group consisting of a direct bond, C₁-C₃ alkyl and C₁-C₃ alkoxy; and R is H, linear or branched C₁-C₆ alkyl, linear or branched C₁-C₆ alkoxy, C₆-C₁₀ aryl and C₅-C₁₀ heteroaryl. In some embodiments, the C₁₆-C₂₀ (poly)unsaturated fatty acid includes, but are not limited to, α-linolenic acid, stearidonic acid, eicosapentaenoic acid, cervonic acid, linoleic acid, linolelaidic acid, arachidonic acid, docosatetraenoic acid, palmitoleic acid, vaccenic acid, oleic acid, elaidic acid, erucic acid, nervonic acid, mead acid or paullinic acid; preferably oleic acid and linolenic acid (such as α- or γ-linolenic acid). In some further embodiments, L and the C₁₆-C₂₀ (poly)unsaturated fatty acid are any combinations of the above listed species of L and the C₁₆-C₂₀ (poly)unsaturated fatty acid.

In some embodiments, L is -A-NR—C(═Y)—B— and -A-C(═Y)—NR—B—, wherein Y is O; A is a direct bond, B is a direct bond and R is H and R¹ and R² are H.

In some embodiments, L is -A-NR—C(═Y)—B— and -A-C(═Y)—NR—B—, wherein Y is O; A is a direct bond, B is a direct bond and R is H and R¹ and R² are each independently oleic acid and linolenic acid (such as α- or γ-linolenic acid).

In some embodiments, L is -A-NR—C(═Y)—B— and -A-C(═Y)—NR—B—, wherein Y is O; A is a direct bond, B is a direct bond and R is linear or branched C₁-C₆ alkyl and R¹ and R² are each H.

In some embodiments, L is -A-NR—C(═Y)—B— and -A-C(═Y)—NR—B—, wherein Y is O; A is a direct bond, B is a direct bond and R is linear or branched C₁-C₆ alkyl and R¹ and R² are each independently oleic acid and linolenic acid (such as α- or γ-linolenic acid).

In some embodiments, L is -A-NR—C(═Y)—B— and -A-C(═Y)—NR—B—, wherein Y is O; A is a direct bond, B is a direct bond and R is C₆-C₁₀ aryl (such as phenyl or benzyl) and R¹ and R² are each H.

In some embodiments, L is -A-NR—C(═Y)—B— and -A-C(═Y)—NR—B—, wherein Y is O; A is a direct bond, B is a direct bond and R is C₆-C₁₀ aryl (such as phenyl or benzyl) and R¹ and R² are each independently oleic acid and linolenic acid (such as α- or γ-linolenic acid).

In some embodiments, L is -A-NR—C(═Y)—B— and -A-C(═Y)—NR—B—, wherein Y is O; A is a direct bond, B is a direct bond and R is linear or branched C₁-C₆ alkyl optionally substituted by C₆-C₁₀ aryl; and R¹ and R² are each H.

In some embodiments, L is -A-NR—C(═Y)—B— and -A-C(═Y)—NR—B—, wherein Y is O; A is a direct bond, B is a direct bond and R is linear or branched C₁-C₆ alkyl optionally substituted by C₆-C₁₀ aryl; and R¹ and R² are each independently oleic acid and linolenic acid (such as α- or γ-linolenic acid).

In one embodiment, the present disclosure provides a compound of Formula (Ia) or (Ib),

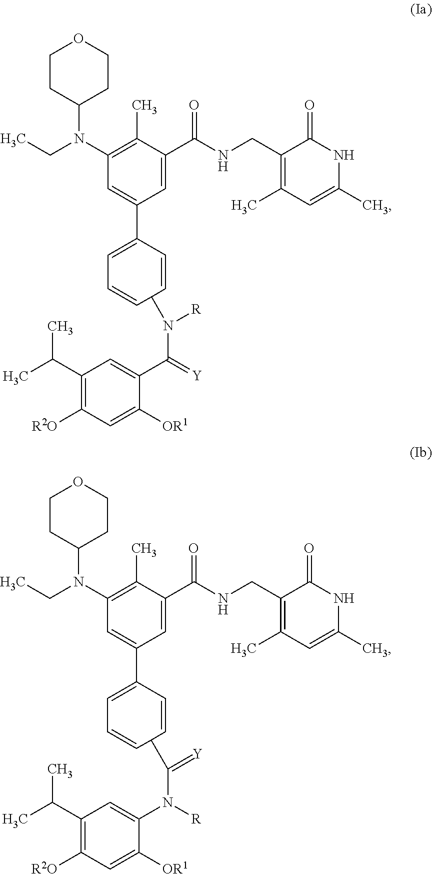

or a pharmaceutically acceptable salt, hydrate, solvate, prodrug or isomer thereof,
wherein:
Y is O or S;
R is H, linear or branched C₁-C₄ alkyl, phenyl or benzyl; and
R¹ and R² are each independently selected from H, halo, C₁-C₆ alkyl and C₁₆-C₂₀ (poly)unsaturated fatty acids, C₁₆-C₂₀ (poly)unsaturated fatty acids preferably being oleic acid and α-linolenic acid;
wherein optionally the linear or branched C₁-C₄ alkyl is substituted by halogen, hydroxy or amino; and
wherein optionally the phenyl or benzyl is substituted by C₁-C₂ alkyl, halogen, hydroxy or amino.

In one embodiment, the present disclosure also provides a compound of Formula (Ia-p) or (Ib-p),

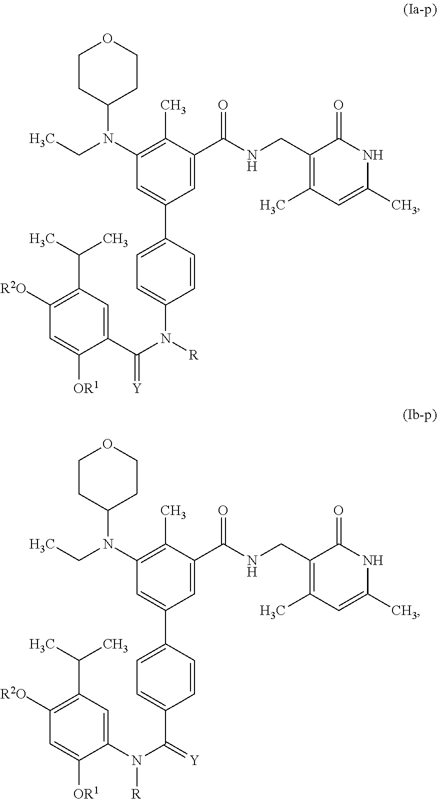

or a pharmaceutically acceptable salt, hydrate, solvate, prodrug or isomer thereof,
wherein:
Y is O or S;
R is H, linear or branched C₁-C₄ alkyl, phenyl or benzyl; and
R¹ and R² are independently selected from H, halo, C₁-C₆ alkyl and C₁₆-C₂₀ (poly)unsaturated fatty acids, preferably oleic acid and α-linolenic acid;
wherein optionally the linear or branched C₁-C₄ alkyl is substituted by halogen, hydroxy or amino; and
wherein optionally the phenyl or benzyl is substituted by C₁-C₂ alkyl, halogen, hydroxy or amino.

In one embodiment, the present disclosure also provides a compound of Formula (Ia-m) or (Ib-m),

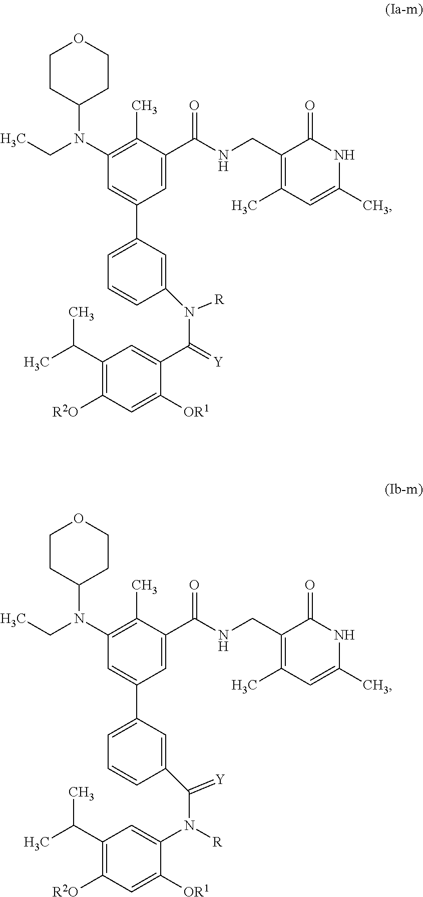

or a pharmaceutically acceptable salt, hydrate, solvate, prodrug or isomer thereof,
wherein:
Y is O or S;
R is H, linear or branched C₁-C₄ alkyl, phenyl or benzyl; and
R¹ and R² are independently selected from H, halo, C₁-C₆ alkyl and C₁₆-C₂₀ (poly)unsaturated fatty acids, preferably oleic acid and α-linolenic acid;
wherein optionally the linear or branched C₁-C₄ alkyl is substituted by halogen, hydroxy or amino; and
wherein optionally the phenyl or benzyl is substituted by C₁-C₂ alkyl, halogen, hydroxy or amino.

In some embodiments of any formula described herein, Y is O and R is H, linear or branched C₁-C₄ alkyl, phenyl or benzyl.

In some embodiments of any formula described herein, Y is O; R is H, linear or branched C₁-C₄ alkyl, phenyl or benzyl; and R¹ and R² are each H; wherein the linear or branched C₁-C₄ alkyl is optionally substituted by halogen, hydroxy, amino or phenyl; and wherein the phenyl or benzyl as R is optionally substituted by C₁-C₂ alkyl, halogen, hydroxy or amino. In some embodiments of any formula described herein, Y is O; R is H, linear or branched C₁-C₄ alkyl, phenyl or benzyl; and R¹ and R² are independently selected from C₁₆-C₂₀ (poly)unsaturated fatty acids; wherein the linear or branched C₁-C₄ alkyl is optionally substituted by halogen, hydroxy, amino or phenyl; and wherein the phenyl or benzyl as R is optionally substituted by C₁-C₂ alkyl, halogen, hydroxy or amino. In some further embodiments of any formula described herein, Y is O; R is H, linear or branched C₁-C₄ alkyl, phenyl or benzyl; and R¹ and R² are each H; wherein the linear or branched C₁-C₄ alkyl is optionally substituted by halogen, hydroxy or amino and wherein the phenyl or benzyl is optionally substituted by C₁-C₂ alkyl, halogen, hydroxy or amino. In some further embodiments of any formula described herein, Y is O; R is H, linear or branched C₁-C₄ alkyl, phenyl or benzyl; and R¹ and R² are independently selected from linolenic acid (such as α- or γ-linolenic acid), stearidonic acid, eicosapentaenoic acid, cervonic acid, linoleic acid, linolelaidic acid, arachidonic acid, docosatetraenoic acid, palmitoleic acid, vaccenic acid, oleic acid, elaidic acid, erucic acid, nervonic acid, mead acid or paullinic acid; preferably oleic acid and α-linolenic acid; wherein the linear or branched C₁-C₄ alkyl is optionally substituted by halogen, hydroxy or amino and wherein the phenyl or benzyl is optionally substituted by C₁-C₂ alkyl, halogen, hydroxy or amino. In some further embodiments of any formula described herein, Y, R, R¹ and R² are any combinations of the above listed species thereof.

In some embodiments of any formula described herein, Y is S and R is H, linear or branched C₁-C₄ alkyl, phenyl or benzyl.

In some embodiments of any formula described herein, Y is S; R is H, linear or branched C₁-C₄ alkyl, phenyl or benzyl; and R¹ and R² are each H; wherein the linear or branched C₁-C₄ alkyl is optionally substituted by halogen, hydroxy or amino and wherein the phenyl or benzyl is optionally substituted by C₁-C₂ alkyl, halogen, hydroxy or amino. In some embodiments of any formula described herein, Y is S; R is H, linear or branched C₁-C₄ alkyl, phenyl or benzyl; and R¹ and R² are independently selected from C₁₆-C₂₀ (poly)unsaturated fatty acids; wherein the linear or branched C₁-C₄ alkyl is optionally substituted by halogen, hydroxy or amino and wherein the phenyl or benzyl is optionally substituted by C₁-C₂ alkyl, halogen, hydroxy or amino. In some further embodiments of any formula described herein, Y is S; R is H, linear or branched C₁-C₄ alkyl, phenyl or benzyl; and R¹ and R² are each H and wherein the phenyl or benzyl is optionally substituted by C₁-C₂ alkyl, halogen, hydroxy or amino. In some further embodiments of any formula described herein, Y is S; R is H, linear or branched C₁-C₄ alkyl, phenyl or benzyl; and R¹ and R² are independently selected from linolenic acid (such as α- or γ-linolenic acid), stearidonic acid, eicosapentaenoic acid, cervonic acid, linoleic acid, linolelaidic acid, arachidonic acid, docosatetraenoic acid, palmitoleic acid, vaccenic acid, oleic acid, elaidic acid, erucic acid, nervonic acid, mead acid or paullinic acid; preferably oleic acid and α-linolenic acid; wherein the linear or branched C₁-C₄ alkyl is optionally substituted by halogen, hydroxy or amino and wherein the phenyl or benzyl is optionally substituted by C₁-C₂ alkyl, halogen, hydroxy or amino. In some further embodiments of any formula described herein, Y, R, R¹ and R² are any combinations of the above listed species thereof.

The present disclosure also provides a process of preparing the compound of formula (I), formula (Ia), formula (Ib), formula (Ia-p), formula (Ib-p), formula (Ia-m) or formula (Ib-m).

Particular examples of the compounds described herein include, but are not limited to:

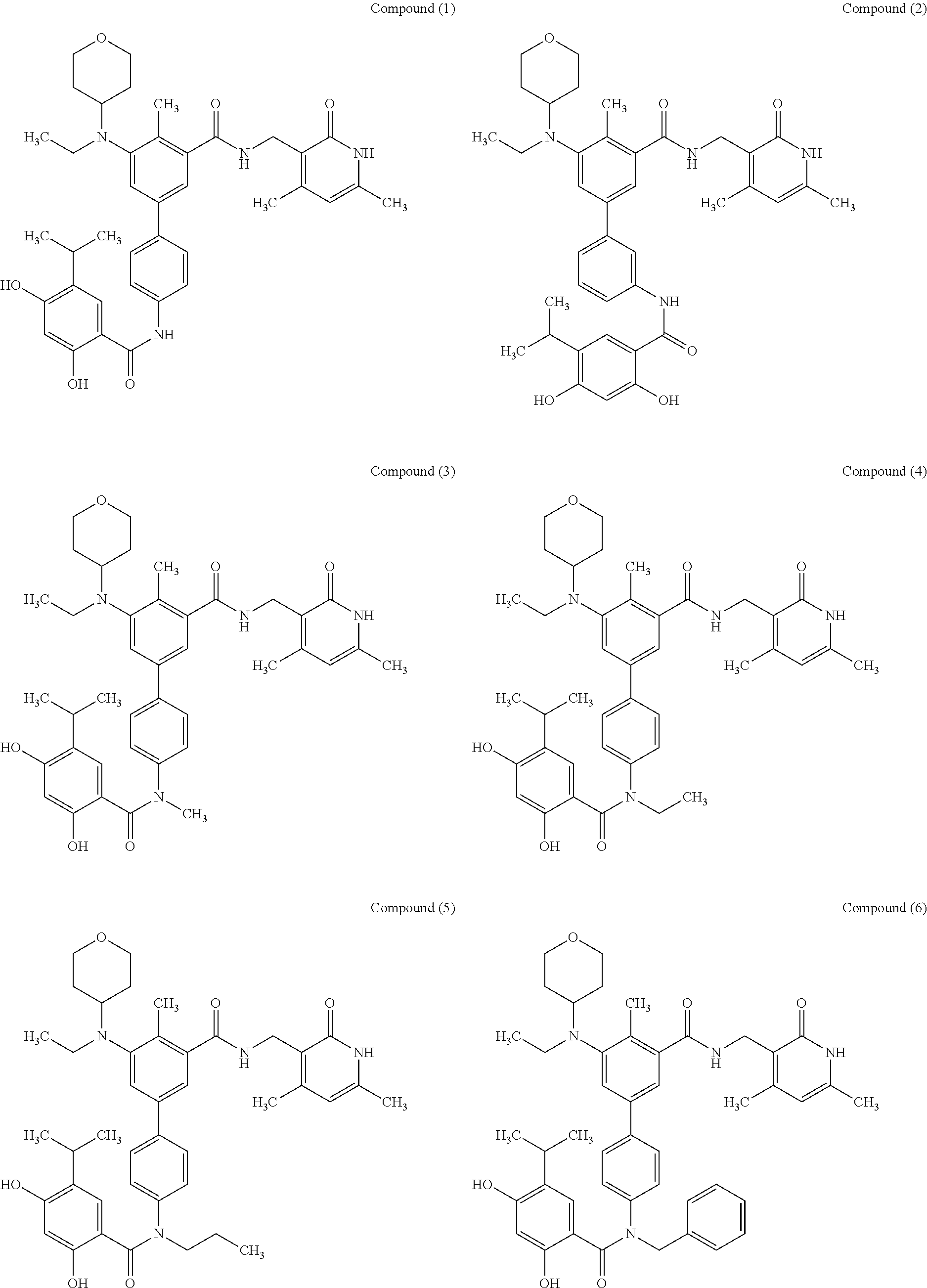

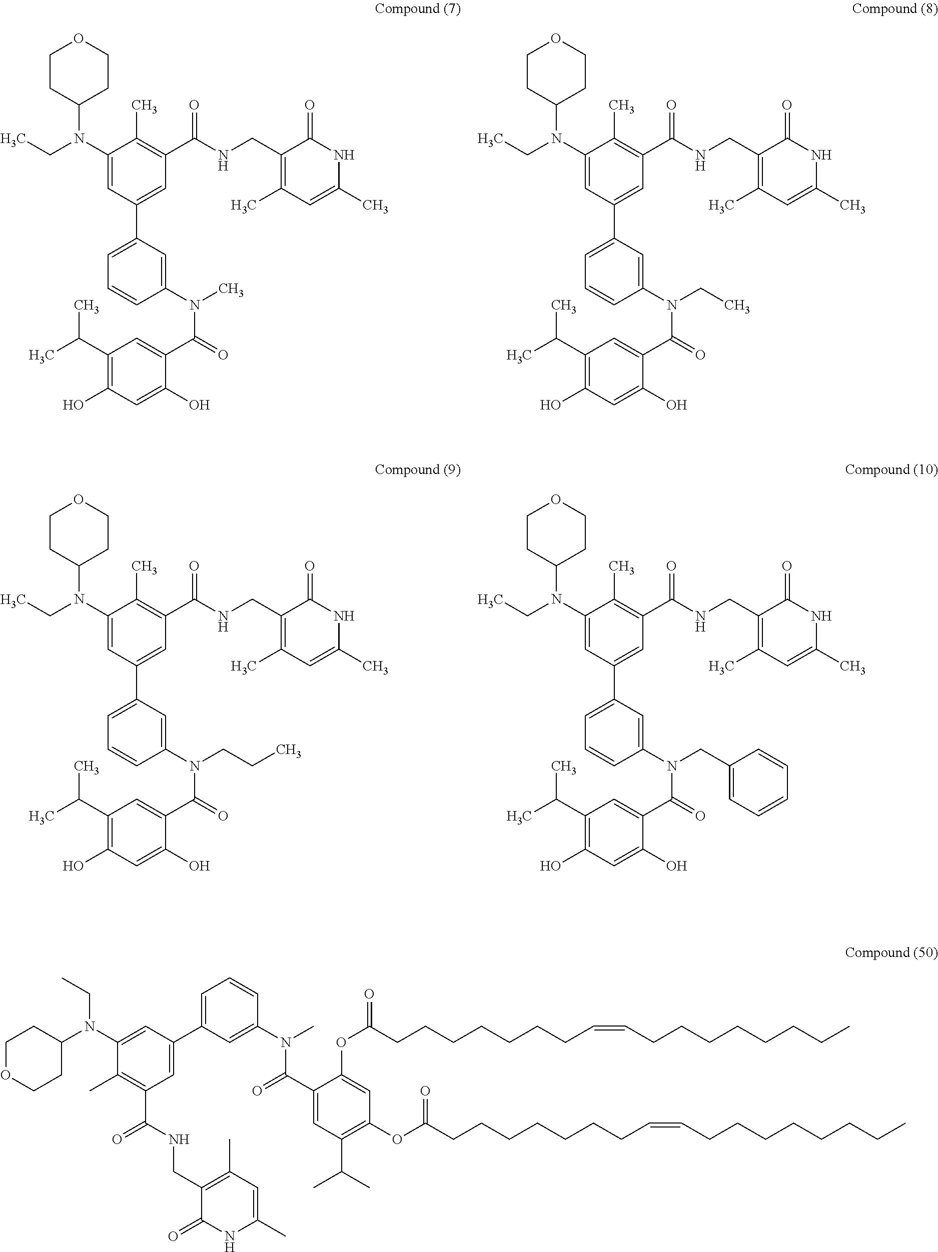

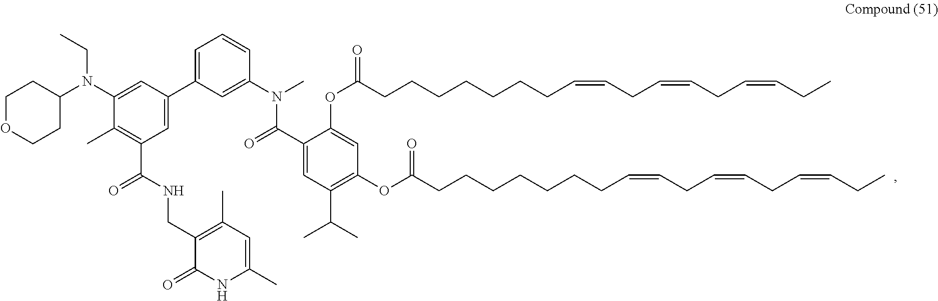 [SMILES: CC/C=C\C/C=C\C/C=C\CCCCCCCC(=O)Oc1cc(OC(=O)CCCCCCC/C=C\C/C=C\C/C=C\CC)c(C(C)C)cc1C(=O)N(C)c1cccc(-c2cc(C(=O)NCc3c(C)cc(C)[nH]c3=O)c(C)c(N(CC)C3CCOCC3)c2)c1]

or a pharmaceutically acceptable salt, hydrate, solvate, prodrug or isomer thereof.

The present disclosure provides a pharmaceutical composition/formulation comprising a compound described herein and a pharmaceutically acceptable carrier.

The present disclosure also provides a method of treating a disease or disorder mediated by EZH2 and/or HSP90, comprising administering a therapeutically effective amount of the compound of formula (I), formula (Ia), formula (Ib), formula (Ia-p), formula (Ib-p), formula (Ia-m) or formula (Ib-m), or a pharmaceutically acceptable salt, hydrate, solvate or prodrug of any of the foregoing, in particular to a subject in need thereof. The present disclosure also provides a pharmaceutical composition/formulation for use in a method for treating a disease or disorder mediated by EZH2 and/or HSP90, wherein the pharmaceutical composition/formulation comprises the compound of formula (I), formula (Ia), formula (Ib), formula (Ia-p), formula (Ib-p), formula (Ia-m) or formula (Ib-m), or a pharmaceutically acceptable salt, hydrate, solvate or prodrug of any of the foregoing.

In one embodiment, the disease or disorder mediated by EZH2 and/or HSP90 is a brain cancer.

In a further embodiment, the brain cancer is a primary brain tumor or a metastatic brain cancer. In some embodiments, the brain cancer is glioma or meningioma.

In a further embodiment, the brain cancer is glioblastoma (GBM).

### BRIEF DESCRIPTIONS OF THE DRAWINGS

FIG. 1 shows EZH2 inhibitors with anti-GBM activity.

FIG. 2 shows structure-based molecular docking of compound 7.

FIG. 3 shows effects of Epz-6438-derived inhibitors on viability of Pt3R cells.

FIGS. 4A-4C show effect of compound 7 on gene expression profile in TMZ-resistant Pt3R cells.

FIGS. 5A-5E show that Compound 7 suppresses gene expression of CENP family.

FIGS. 6A-6H show that Compound 7 suppresses DNA repair-related genes.

FIGS. 7A-7C show that Compound 7 triggers ROS production from mitochondria.

FIGS. 8A and 8B show the effect of compound 7 on the growth of TMZ-resistant Pt3R cells in vivo.

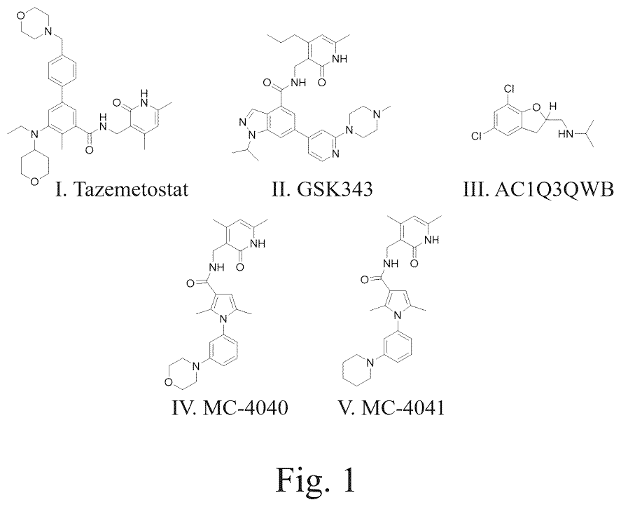

### DETAILED DESCRIPTION OF THE INVENTION

In order to facilitate understanding of the disclosure herein, terms as used herein are hereby defined below.

In the context of the specification and the claims, the singular forms “a,” “an” and “the” include plural referents, unless specifically indicated otherwise. Unless otherwise stated, any and all examples or exemplary language (e.g., “such as”) provided herein is merely used for better illustration of the present invention, instead of limiting the scope of the present invention.

It is to be understood that any numerical range recited in this specification is intended to include all sub-ranges encompassed therein. For example, a range from “50 to 70 °C” includes all sub-ranges and specific values between the stated minimum value of 50 °C and the stated maximum value of 70 °C, inclusive, e.g. from 58 °C to 67 °C, and from 53 °C to 62 °C, 60 °C or 68 °C. Since the numerical ranges disclosed are continuous, they contain each numerical value between the minimum and maximum value. Unless otherwise specified, the various numerical ranges indicated in this specification are approximate.

The term “about” refers to an acceptable deviation of a given value measured by a person of ordinary skill in the art, depending, in part, on how to measure or determine the value.

The term “hydrocarbyl” as used herein refers to a mono-valent radical derived from hydrocarbons. The term “hydrocarbon” as used herein refers to a molecule that consists of carbon and hydrogen atoms only. Examples of hydrocarbons include, but are not limited to, (cyclo)alkanes, (cyclo)alkenes, alkadienes, aromatics, etc. When the hydrocarbyl is further substituted as mentioned above, the substituent can be halogens, amino groups, a hydroxy group, a thiol group, etc. When the hydrocarbyl is interrupted with a heteroatom as mentioned above, the heteroatom can be S, O or N. In the present invention, a hydrocarbyl preferably comprises to 30 C atoms.

The term “alkyl” refers to a saturated, straight or branched alkyl, which comprises preferably 1-10 carbon atoms, and more preferably 1-4 carbon atoms. Examples of alkyl include, but are not limited to, methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, isoheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, 1,1,3,3,5,5-hexamethylhexyl, or the like.

The term “alkoxyl” or “alkoxy” as used herein means a group having a formula “—O-alkyl,” wherein the definition of the “alkyl” in said formula has the meaning of “alkyl” as stated above.

The term “cycloalkyl” as used herein means a saturated or partially unsaturated cyclic carbon radical containing 3 to 10 ring carbon atoms and more preferably 3 to 8 ring carbon atoms, and optionally an alkyl substituent(s) on the ring. Examples of cycloalkyl include, but are not limited to, cyclopropyl, cyclopropenyl, cyclobutyl, cyclopentyl, cyclohexyl, 2-cyclohexen-1-yl, and the like.

The term “aryl” as used herein means a radical comprising at least one aromatic moiety, preferably having 6 to 10 carbon atoms. The aromatic moiety may be a single ring or fused multiple rings. Examples of aryl include, but are not limited to, phenyl, benzyl, tolyl, xylyl(s), indenyl, naphthyl, mesitylenyl, durenyl, and the like. Preferably, the aryl is phenyl or benzyl, or more preferably benzyl. The term “heteroaryl” as used herein means a radical comprising at least one aromatic moiety that has at least one heteroatom selected from the group consisting of oxygen, nitrogen and sulfur, preferably having 5 to 10 carbon atoms. Examples of heteroaryl include, but are not limited to, furanyl, pyrrolyl, diazolyl(s) (e.g., imidazolyl, pyrazolyl), thiophenyl, pyranyl, pyridinyl, diazinyl(s) (e.g., pyridazinyl, pyrimidinyl, pyrazinyl), oxazolyl, isoxazolyl, thiazolyl, isothiazolyl, isobenzofuranyl, quinolinyl, isoquinolinyl, indazolyl, indolyl, isoindolyl, etc.

The term “halogen” or “halo” denotes fluorine, chlorine, bromine or iodine.

The term “amino” as used herein means a functional group of the formula —NRₐR_{b}, wherein Ra and R_{b} each independently represent hydrogen or a hydrocarbyl group as defined above.

The term “(poly)unsaturated fatty acid” as used herein means a fatty acid comprising at least one double bond in the main chain. Examples of the C₁₆-C₂₀ (poly)unsaturated fatty acid include, but are not limited to, hexadecatrienoic acid (HTA), α-linolenic acid (ALA), stearidonic acid (SDA), linoleic acid (LA), γ-linolenic acid (GLA), oleic acid, rumenic acids, α-calendic acid, β-calendic acid, jacaric acid, α-eleostearic acid, β-eleostearic acid, catalpic acid, punicic acid, rumelenic acid, α-parinaric acid, β-parinaric acid, pinolenic acid, eicosatrienoic acid (ETE), eicosatetraenoic acid (ETA), eicosapentaenoic acid (EPA), eicosadienoic acid, dihomo-γ-linolenic acid (DGLA), arachidonic acid (AA), eicosenoic acids and mead acid. In certain embodiments, the C₁₆-C₂₀ (poly)unsaturated fatty acids are selected from oleic acid and linolenic acid.

The term “therapeutically acceptable salt” refers to salts or zwitterions of pharmaceutical compounds which are water or oil-soluble or dispersible, suitable for treatment of disorders and effective for their intended use. The salts may be prepared, for instance, during the final isolation and purification of the compounds or separately by reacting an amino group of the compounds with a suitable acid.

The term “treatment” as used herein includes the alleviation, prevention, reversal, amelioration or control of a pathology, disease, disorder, process, condition or event, such as diabetes, or the symptoms of such pathology, disease, disorder, process, condition or event.

The term “prodrug” refers to compounds that are transformed in vivo to a pharmaceutical compound, for example, by hydrolysis in blood. The term “prodrug” refers to compounds that contain, but are not limited to, substituents known as “therapeutically suitable esters.” In certain embodiments, the “prodrug” relates to the compounds being modified with or having a (poly)unsaturated fatty acid moiety in the structure, e.g., the compounds wherein R¹ and/or R² is C₁₆-C₂₀ (poly)unsaturated fatty acids.

There is an abundance of evidence that the overexpression of EZH2 in cancer stem cells of malignant tumors plays a critical role in cancer stem cell expansion and maintenance (Wen, Y.; Cai, J.; Hou, Y.; Huang, Z.; Wang, Z. Role of EZH2 in cancer stem cells: from biological insight to a therapeutic target. Oncotarget 2017, 8 (23), 37974; Zhang, H.; Qi, J; Reyes, J. M.; Li, L.; Rao, P. K; Li, F.; Lin, C. Y.; Perry, J. A.; Lawlor, M. A.; Federation, A. Oncogenic Deregulation of EZH2 as an Opportunity for Targeted Therapy in Lung Cancer An Oncogenic Role for EZH2 in Lung Cancer. Cancer Discov. 2016, 6 (9), 1006-1021). EZH2, a crux subunit of the Polycomb Repressive Complex (PRC2), is responsible for methylating lysine 27 (mono-, di- and trimethylation) in histone H3 (H3K27), and H3K27me3 is more frequently interlinked with transcriptional repression (Zhuang, S. Histone methyltransferase EZH2: a potential therapeutic target for kidney diseases. Front. Physiol. 2021, 12, 640700; Hill, M. A.; Kollak, I.; Keck, M.; Schroeder, V.; Wirth, J.; Skronska-Wasek, W.; Schruf, E.; Strobel, B.; Stahl, H. An EZH2-dependent transcriptional complex promotes aberrant epithelial remodelling after injury. EMBO reports 2021, 22 (8), e52785). Resultantly, several key revelations have been made, such as i) involvement of miR-206/Twist axis in EZH2-regulated malignancy of GBM cells, ii) mediation of pro-oncogenic actions of E2 (proliferation, migration, and invasion) by EZH2 in GBM cells, iii) direct transcriptional regulation of c-myc by EZH2 leading to the maintenance of GSCs.

Despite the well-explored role of EZH2 in diverse malignancies, there is only one EZH2 inhibitor, Tazemetostat, approved by the FDA in 2020 for treating epithelioid sarcoma and follicular lymphoma (Huang, N.; Liao, P.; Zuo, Y.; Zhang, L.; Jiang, R. Design, Synthesis, and Biological Evaluation of a Potent Dual EZH2-BRD4 Inhibitor for the Treatment of Some Solid Tumors. J. Med. Chem. 2023).

The present disclosure modulates the histone deacetylase (HDAC) inhibitory structural template to outwit the pharmacodynamics and physicochemical-related liabilities associated with HDAC inhibitors in pursuit of extracting anti-GBM efficacy (Hsu, C.-C.; Chang, W.-C.; Hsu, T.-L.; Liu, J.-J.; Yeh, S.-H.; Wang, J.-Y.; Liou, J.-P.; Ko, C.-Y.; Chang, K.-Y.; Chuang, J.-Y. Suberoylanilide hydroxamic acid represses glioma stem-like cells. J. Biomed. Sci. 2016, 23 (1), 1-12; Nepali, K.; Wu, A.-C.; Lo, W.-L.; Chopra, B.; Lai, M.-J.; Chuang, J.-Y.; Liou, J.-P. Rationally designed donepezil-based hydroxamates modulate Sig-1R and HDAC isoforms to exert anti-glioblastoma effects. Eur. J. Med. Chem. 2023, 115054). Accordingly, the present disclosure relates to candidature of EZH2 as an epigenetic target for constructing new anti-GBM chemical architectures. Tracking the progress of the cocktail of EZH2 inhibitors in GBM at the clinical level did not confer or reveal a starting point for selecting another target. The present disclosure thus aims to a “campaign running” to evaluate the anticancer efficacy of cocktails of epigenetic inhibitors and mechanistically diverse agents. Notably, the campaign supports us in designing dual targeting adducts on the basis of evidenced cell growth inhibitory effects of the combinations evaluated.

Delightfully, the results of the campaign drew the inventors' attention towards the magnificent, remarkable cell growth inhibitory effects attained with the combination of Tazemetostat (EZH2 inhibitor) and STA9090 (HSP90 inhibitor) against GBM cell lines, and this served as the point of inception for this endeavour. It was intriguingly alluring that tazemetosat demonstrated cytotoxicity-devoid trends against GBM cell lines. Despite this disappointing trend witnessed with tazemetostat, precedential evidence (preclinical study) of extracting anti-GBM efficacy of tazemetostat via concomitant administration with PI3K inhibitors provided us sufficient motivation to employ the strategy of designing bifunctional EZH2 inhibitory adducts (dual inhibitors). This approach was driven by anticipation that, similar to combination therapy, simulation modulation of an additional target along with EZH2 via dual inhibitors might activate the chemical architecture of tazemetostat to manifest anti-GBM efficacy.

Unlike Tazemetostat, STA9090 as an individual component of the cocktail exhibited moderate cytotoxicity against GBM cell lines. However, the cell growth inhibitory effects were several fold less than that observed with the cocktail. As such, Hsp90 is an ATP-dependent molecular chaperone that regulates protein conformation, stability, and degradation. Several HSP90 inhibitors have been evaluated for their anti-GBM efficacy at a preliminary level. For instance, AUY922, a resorcinol-based HSP90 inhibitor demonstrated magnificent, impressive anti-GBM efficacy and led to GBM cell death via apoptosis and autophagy. Reduction in the mRNA and protein expression of EGFR, PDGFRA, CDK4, and NF1 in heterogeneous GBM cells was also evidenced in treatment with AUY922. Another HSP90 inhibitor, YZ129, promoted apoptosis and exerted GBM cell cycle arrest at the G2/M phase, inhibiting tumour cell proliferation and migration. Also, a pochoxime-based HSP90 inhibitor, NW457, with a favourable brain pharmacokinetic profile, demonstrated the ability to ameliorate the therapeutic outcome of fractionated CBCT-based irradiation in an orthotopic, syngeneic GBM mouse model. Another study reported the potential of HSP90 inhibitor, BIIB021, to overcome resistance to molecular targeted therapy in brafv600e mutant GBM.

The present disclosure thus envisions that EZH2 and HSP90 inhibition might act through a unique pathway responsible for the observed anti-proliferative effects against GBM cell lines. However, aligned with the current perspective of the medicinal chemist, rather than proposing conjecture regarding a biochemical correlation between the two targets, the present disclosure plans to accentuate the positive aspects of the preliminary work program and validate the preliminary evidence via the design of dual EZH2-HSP90 inhibitory chemical probes.

The present disclosure is generally directed to small molecule compounds of dual EZH2-HSP90 inhibitors and their uses as effective therapeutics. These compounds can be administered to treat/control/mitigate diseases and conditions of cancers; preferably brain cancers.

Embodiments of the compounds are described in the Summary of the Invention section, and preparations thereof are illustrated in the examples. Persons of ordinary skill can prepare the compounds according to the teachings of the examples.

The compound of the present disclosure can be prepared as a pharmaceutical composition or formulation for administrating to a subject to treat a cancer such as a brain cancer. Examples of the brain cancer includes, bur are not limited to, primary brain cancers or metastatic brain cancers, gliomas, meningiomas and glioblastomas. In one embodiment, the pharmaceutical composition of the invention comprises a second anti-cancer agent.

While it may be possible for the compounds of the present disclosure to be administered as the raw chemical, it is also possible to present them as a pharmaceutical composition or formulation. Accordingly, the present disclosure provides a pharmaceutical formulation or composition comprising a compound or a pharmaceutically acceptable salt, prodrug or solvate thereof, together with one or more pharmaceutically acceptable carriers thereof and optionally one or more other therapeutic ingredients. The carrier(s) must be “acceptable” in the sense of being compatible with the other ingredients of the composition or formulation and not deleterious to the recipient thereof. Proper composition/formulation is dependent upon the route of administration chosen. Compositions/formulations may take the form of tablets, pills, capsules, semisolids, powders, sustained release formulations, solutions, suspensions, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions/formulations, may be found in such standard references as Remington: The Science and Practice of Pharmacy, A. Gennaro, ed., 20th edition, Lippincott, Williams & Wilkins, Philadelphia, Pa. Suitable liquid carriers, especially for injectable solutions, include water, aqueous saline solution, aqueous dextrose solution, and glycols. The pharmaceutical compositions/formulations of the present disclosure may be manufactured in a manner that is itself known, e.g., by means of conventional mixing, dissolving, granulating, dragee-making, levigating, emulsifying, encapsulating, entrapping or compression processes.

The compositions/formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration, although the most suitable route may depend, for example, upon the condition and disorder of the recipient. Oral administration is a preferred route. The compositions/formulations may conveniently be presented in unit dosage form and may be prepared by any of the methods well known in the art of pharmacy. All methods include the step of bringing into association a compound of the present invention or a pharmaceutically acceptable salt, prodrug or solvate thereof (“active ingredient”) with the carrier which constitutes one or more accessory ingredients. In general, the compositions/formulations are prepared by uniformly and intimately bringing into association the active ingredient with liquid carriers or finely divided solid carriers or both and then, if necessary, shaping the product into the desired composition/formulation.

For oral administration, suitable pharmaceutical compositions/formulations of the present disclosure include powders, granules, pills, tablets, lozenges, chews, gels, and capsules as well as liquids, syrups, suspensions, elixirs, and emulsions. These compositions/formulations may also include anti-oxidants, flavorants, preservatives, and suspending, thickening and emulsifying agents, colorants, flavoring agents and other pharmaceutically acceptable additives. Formulations for oral administration may be formulated to be immediate release or modified release, where modified release includes delayed, sustained, pulsed, controlled, targeted and programmed release.

For parenteral administration, the compounds or compositions/formulations of the present disclosure are administered directly into the blood stream, into muscle, or into an internal organ via an intravenous, intraarterial, intraperitoneal, intramuscular, subcutaneous or other injection or infusion. Parenteral formulations may be prepared in aqueous injection solutions which may contain, in addition to the compound of the invention, buffers, antioxidants, bacteriostats, salts, carbohydrates, and other additives commonly employed in such solutions. Parenteral administrations may be immediate release or modified release (such as an injected or implanted depot).

Compounds or compositions/formulations of the present disclosure may also be administered topically, (intra)dermally, or transdermally to the skin or mucosa. Typical formulations include gels, hydrogels, lotions, solutions, creams, ointments, dressings, foams, skin patches, wafers, implants and microemulsions. Compounds or compositions/formulations of the present invention may also be administered via inhalation or intranasal administration, such as with a dry powder, an aerosol spray or as drops. Additional routes of administration for compounds of the present invention include intravaginal and rectal (by means of a suppository, pessary or enema), and ocular and aural.

The following examples are provided to make the present disclosure more comprehensible to those of ordinary skill in the art to which the present disclosure pertains, but are not intended to limit the scope of the invention.

### EXAMPLES

### Experiments and Materials

Chemistry Nuclear magnetic resonance (¹H NMR and ¹³C NMR) spectra were obtained with a Bruker DRX-500 spectrometer operating at 300 MHz. Chemical shifts are reported in parts per million (ppm, 6) downfield from TMS as an internal standard. High-resolution mass spectra (HRMS) were measured with a JEOL (JMS-700) electron impact (EI) mass spectrometer. The purity of the final compounds was determined using a Hitachi 2000 series HPLC system using C-18 column (Agilent ZORBAX Eclipse XDB-C18 5 mm. 4.6 mm 150 mm). Column chromatography was accomplished on silica gel (Merck Kieselgel 60, No. 9385, 230e400 mesh ASTM). All reactions were carried out under an atmosphere of dry nitrogen.

### EZH2 Inhibition Assay

The EZH2 inhibitory ability of the synthesized adduct was assessed by Reaction Biology Corporation, Malvern, PA (http://www.reactionbiology.com).

### In Vitro Hsp90 Assay

The HSP90 inhibitory activity of the synthesized adduct was assessed by Reaction Biology Corporation, Malvern, PA (http://www.reactionbiology.com).

### Cell Culture

Pt3 GBM cells were isolated from a male GBM patient as described in our previous study. Temozolomide (TMZ)-resistant phenotype of Pt3R cells had been validated previously. Pt3 cells were cultured in DMEM-supplemented with 10% fetal bovine serum, 100 μg/ml streptomycin and 100 μg/ml penicillin G. Pt3R cells were cultured in the presence of 100 μM of TMZ (MilliporeSigma Corporate, St. Louis, MO, USA).

### Cell Viability CCK8 Assay

The CCK8 reagent was purchased from TargetMOI (Wellesley Hills, MA, USA) and used according to the manufacturer's instruction.

### Molecular Docking

Structure-based molecular docking was performed to screen the MPT1A059 based on the binding energy. The docking process was carried out between protein and compounds with a population size of 1500, generations of 150, and solutions of 10 through iGEMDOCK version 2.1. Protein structures of HSP90 and EZH2 were obtained from Protein Data Bank (8AGI and 4W2R).

### RNA-Seq and Proteomic Assay

RNA was isolated using the RNA extracting kit (Zymo Research, Irvine, CA, USA), and subjected to RNA-seq serviced by BIOTOOLS Co., Ltd (New Taipei City, Taiwan). Cell pellets were collected and subjected to proteomic analysis serviced by BIOTOOLS Co., Ltd. The comparison in gene expression between Pt3 and Pt3R was performed using RNA seq, and has been described previously [60].

### ROS Assay and MitoSOX Assay

Dihydrorhodamine 123 (DHR; Thermo Fisher Scientific, Waltham, MA, USA) was used to label cellular ROS detected by flow cytometry [54], and the MitoSOX reagent (Thermo Fisher Scientific) was used to label mitochondrial ROS [55].

### Western Blotting

The details of the procedure are described in our previous study [54, 55, 58, 59]. Primary antibodies, anti-CENPA, anti-CENPE, anti-CENPF and anti-CENPI antibodies, were purchased from abcam (Cambridge, UK); anti-CDK1 and anti-cyclin B1 antibodies were purchased from Santa Cruz Biotechnology, Inc. (Dallas, TX, USA); anti-EZH2 and anti-H3K27^{me3} antibodies were purchased from GeneTex International Corporation (Hsinchu, Taiwan).

### Bioinformatic Analysis

Gepia and GlioVis websites were employed to generate expression and survival plots.

### Animal Experiment

NOD.CB17-Prkdc^{scid}/NCrCrl mice (8-week-old) were purchased from BioLASCO Taiwan Co., Ltd. (Taipei, Taiwan). Pt3R cells (1×10⁶) in 50 ml DMEM were injected onto the backs of mice. After 10 days, tumor was touchable. Mice were injected with DMSO (the control group) or MPT1A059 (5 mg/kg) twice per week.

### Statistical Analysis

Experiments were performed 3 times independently, and data were expressed as mean±s.e.m. P value <0.05 was considered as the significant difference.

### Synthetic Example 1

Scheme 1 shown below is an example of synthesizing the compounds disclosed herein.

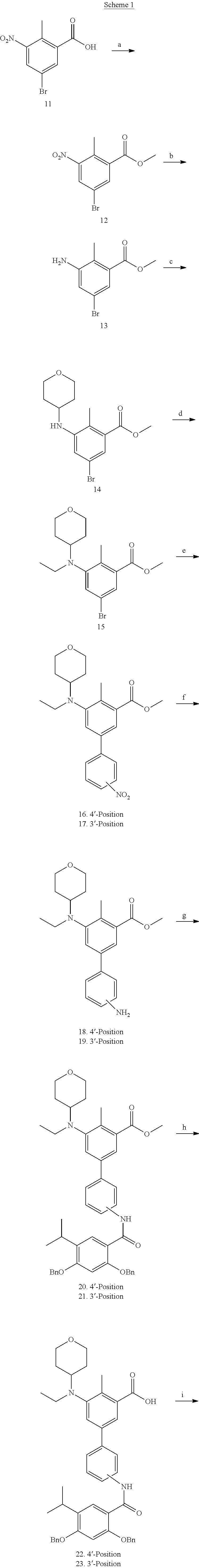

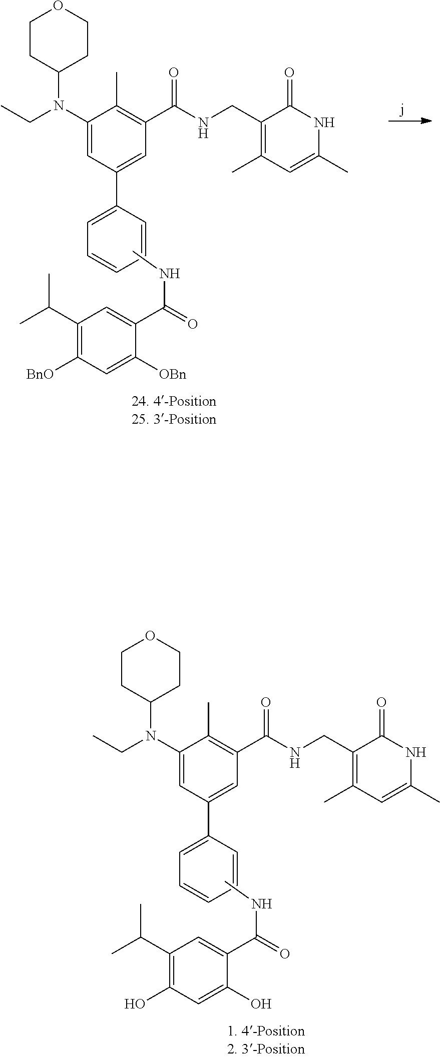

The route commenced with the high-yielding esterification of 5-bromo-2-methyl-3-nitrobenzoic acid 11 to generate the ester 12. The nitro group bearing aromatic ester was then subjected to Fe/NH₄Cl mediated reduction to afford the primary amine 13 which was reductively aminated consecutively, first to produce the pyran bearing intermediate 14 and then to furnish N-ethyl substitution bearing tertiary amine 15. Organopalladium catalyzed Suzuki arylation of intermediate 15 with (4-nitrophenyl)boronic acid, and (3-nitrophenyl)boronic acid generated the biphenyl frameworks 16 and 17. Fe/NH₄Cl mediated nitro reduction transformed the nitro group containing biphenyls 16 and (17) to amine functionality bearing biphenyls 18 and 19. The biphenyls 18 and 19 were further amidated with 2,4-dihydroxy-5-isopropylbenzoic acid employing the carbodiimide-mediated methodology to accomplish the intermediates 20 and 21. Next, the ester functionality located on the trisubstituted phenyl ring of the biphenyl 20 and 21 was hydrolysed using lithium hydroxide to produce the carboxylic acids 22 and 23. The acids were subsequently subjected to EDC/HOBt assisted amidation employing 3-(aminomethyl)-4,6-dimethylpyridin-2(1H)-one as the amine to obtain the intermediates 24 and 25. The intermediates 24 and 25 were then debenzylated using 10% Pd/C in methanol in a hydrogenation vessel with H2 at 40-42 psi to attain the hybrid scaffolds 1-2.

In particular, the reagents and conditions used in Scheme 1 are as follows: a) CH₃I, K₂CO₃, DMF, rt, overnight; b) Fe, NH₄Cl, EtOH:H₂O (9:1), 100 °C, 2 h; c) tetrahydro-4H-pyran-4-one, CH₃COOH, NaBH₃CN, MeOH, reflux, overnight; d) acetaldehyde, CH₃COOH, Na(CH₃COO)₃BH, DCE, rt, 5 h; e) 4-Nitrophenyl boronic acid or 3-Nitrophenyl boronic acid, Pd(PPh₃)₃, Na₂CO₃, dioxane:water (9:1), 60 °C, 2 h; f) Fe, NH₄Cl, EtOH:H₂O (9:1), 100° c, 2 h, g) 2,4-bis(benzyloxy)-5-isopropylbenzoic acid, EDC.HCl, HOBT, DIPEA, DMF, rt, 3 h; h) LiOH(aq), dioxane, rt, 3 h; i) 3-(aminomethyl)-4,6-dimethylpyridin-2(1H)-one, EDC.HCl, HOBT, DIPEA, DMF, rt, 3 h; j) Pd/C, H2, MeOH, rt, 4 h.

### Methyl 5-bromo-2-methyl-3-nitrobenzoate (12)

In a 250 ml round bottom flask (RBF), compound 11 (5 gm, 0.019 mol) and potassium carbonate (5.31 gm, 0.038 mol) were dissolved in 50 ml of DMF, and iodomethane (3.54 gm, 0.024 mol) was added dropwise to the reaction mixture. The resulting mixture was stirred overnight at room temperature and progress of the reaction was monitored using TLC. After completion of the reaction, 100 ml of cold water was added to it and the resulting precipitates were filtered under suction, washed with water and dried. The precipitates were carried to the next step without further purification with a yield of 90%. ¹H NMR (300 MHz, DMSO-d₆): δ 7.31 (s, 1H), 7.27 (s, 1H), 3.87 (s, 3H), 2.26 (s, 3H).

### Methyl 3-amino-5-bromo-2-methylbenzoate (13)

Compound 12 (5 gm, 0.020 mol), iron powder (5.69 gm, 0.102 mol) and ammonium chloride (2.17 gm, 0.040 mol) were weighed in a 100 ml RBF, and a mixture of ethanol and water (18:2 ml) was added as a solvent. The reaction mixture was refluxed at 100 °C for 2 hours and the progress of the reaction was monitored by TLC. After completion of the reaction, the mixture was diluted with methanol and passed through celite. The solvent was evaporated in-vacuo and the residue was purified by silica gel column chromatography (ethyl acetate:hexane: 1:5) with a yield of 91%. ¹H NMR (300 MHz, CDCl₃): δ 7.29 (d, J=2.4 Hz, 1H), 6.91 (d, J=2.4 Hz, 1H), 3.88 (s, 3H), 3.80 (s, 2H), 2.24 (s, 3H).

### Methyl 5-bromo-2-methyl-3-((tetrahydro-2H-pyran-4-yl)amino)benzoate (14)

In a 100 ml RBF, compound 13 (5 gm, 0.020 mol) and tetrahydro-4H-pyran-4-one (4.10 gm, 0.040 mol) were dissolved in 100 ml of methanol, and acetic acid (1.22 gm, 0.020 mol) was added thereto. The mixture was stirred at room temperature for 30 minutes, followed by portion-wise addition of sodium cyanoborohydride (3.83 gm, 0.60 mol). The reaction was refluxed overnight and the progress of the reaction was monitored using TLC. After completion of the reaction, the resulting precipitates were filtered under suction, washed with methanol and dried. The compound was carried to the next step without further purification with a percentage yield of 88%. ¹H NMR (300 MHz, CDCl₃): δ 7.23 (d, J=1.2 Hz, 1H), 6.85 (s, 1H), 3.97-4.03 (m, 2H), 3.86 (s, 3H), 3.46-3.56 (m, 3H), 2.23 (s, 3H) 2.03 (d, J=11.4 Hz, 2H), 1.48-1.54 (m, 2H)

### Methyl 5-bromo-3-(ethyl(tetrahydro-2H-pyran-4-yl)amino)-2-methylbenzoate (15)

In a 100 ml RBF, Compound 14 (3 gm, 0.009 mol), acetaldehyde (0.8 gm, 0.018 mol) and acetic acid (0.54 gm, 0.009 mol) were dissolved in 60 ml of dichloroethane, and the reaction was stirred at room temperature for 30 minutes, followed by portion-wise addition of sodium triacetoxyborohydride (5.72 gm, 0.027 mol) under ice cold conditions. The reaction was continued at room temperature for 5 hours. After completion of the reaction (TLC), the reaction mixture was diluted with cold water (100 ml) and the compound was extracted using ethyl acetate (50 ml×3). The combined organic layer was dried over anhydrous magnesium sulfate and concentrated under reduced pressure. The residue was further purified by performing silica gel column chromatography (ethyl acetate:hexane: 1:5) with a yield of 90%; ¹H NMR (300 MHz, CDCl₃): δ 7.73 (d, J=1.8 Hz, 1H), 7.38 (d, J=1.8 Hz, 1H), 3.94-4.00 (m, 2H), 3.91 (s, 3H), 3.30-3.38 (m, 2H), 3.04-3.11 (m, 2H), 2.81-3.00 (m, 1H), 2.47 (s, 3H), 1.63-1.74 (m, 4H), 0.89 (t, J=6.9 Hz, 3H).

### Methyl 5-(ethyl(tetrahydro-2H-pyran-4-yl)amino)-4-methyl-4′-nitro-[1,1′-biphenyl]-3-carboxylate (16)

In a 100 ml RBF, compound 15 (4 gm, 0.011 mol), corresponding 4-nitro phenyl boronic acid (2.24 gm, 0.015 mol), palladium triphenylphosphine (1.15 gm, 0.001 mol) and sodium carbonate (3.49 gm, 0.033 mol) was dissolved in 40 ml of DMF and the reaction was refluxed at 100 °C for 4 hours. After completion of the reaction (TLC), the reaction mixture was diluted with water (100 ml) and the compound was extracted using ethyl acetate (50 ml×3). The combined organic layer was dried over anhydrous magnesium sulfate and concentrated under reduced pressure. The residue was further purified by performing silica gel column chromatography (ethyl acetate:hexane: 1:3) with a yield of 71%. ¹H NMR (300 MHz, DMSO-d₆): δ 8.31 (d, J=7.2 Hz, 2H), 7.85 (s, 1H), 7.74 (d, J=7.2 Hz, 2H), 7.52 (s, 1H), 3.93-3.95 (m, 5H), 3.02-3.39 (m, 5H), 2.57 (s, 3H), 1.69-1.74 (m, 4H), 0.95 (t, J=6.9 Hz, 3H).

### Methyl 5-(ethyl(tetrahydro-2H-pyran-4-yl)amino)-4-methyl-3′-nitro-[1,1′-biphenyl]-3-carboxylate (17)

Intermediate 17 was synthesized with 70% yield using the same experimental procedure as that described for the synthesis of compound 16. ¹H NMR (300 MHz, CDCl₃): δ 8.41 (t, J=2.1 Hz, 1H), 8.21-8.25 (m, 1H), 7.91-7.93 (m, 1H), 7.86 (d, J=1.8 Hz, 1H), 7.65 (t, J=8.1 Hz, 1H), 7.53 (d, J=1.8 Hz, 1H), 4.02 (s, 1H), 3.97 (s, 4H), 3.33-3.41 (m, 2H), 3.15-3.22 (m, 2H), 2.85-3.10 (m, 1H), 2.60 (s, 3H), 1.69-1.76 (m, 4H), 0.95 (t, J=7.2 Hz, 3H).

### Methyl 4′-amino-5-(ethyl(tetrahydro-2H-pyran-4-yl)amino)-4-methyl-[1,1′-biphenyl]-3-carboxylate (18)

Intermediate 16 (1 gm, 0.0025 mol), iron powder (0.69 gm, 0.012 mol) and ammonium chloride (0.267 gm, 0.005 mol) were weighed in a 100 ml round bottom flask (RBF), and a mixture of ethanol and water (27:3 ml) was added as a solvent. The reaction mixture was refluxed at 100 °C for 2 hours and the progress of the reaction was monitored by TLC. After completion of the reaction, the reaction mixture was diluted with methanol and passed through celite. The resulting solvent mixture was evaporated in-vacuo and the residue was purified by silica gel column chromatography (ethyl acetate:hexane: 1:4) with a yield of 80%. ¹H NMR (300 MHz, CD₃OD): δ 7.22-7.26 (m, 3H), 7.08 (d, J=1.5 Hz, 1H), 6.61 (d, J=8.5 Hz, 2H), 3.91-3.94 (m, 5H), 3.06-3.34 (m, 5H), 2.54 (s, 3H), 1.63-1.69 (m, 4H), 0.93 (t, J=6.9 Hz, 3H).

### Methyl 3′-amino-5-(ethyl(tetrahydro-2H-pyran-4-yl)amino)-4-methyl-[1,1′ biphenyl]-3-carboxylate (19)

Intermediate 19 was synthesized with 74% yield, employing intermediate 17 as the starting material, using the same experimental procedure as that described for the synthesis of compound 18. ¹H NMR (300 MHz, CDCl₃): δ 7.83 (d, J=2.1 Hz, 1H), 7.51 (s, 1H), 7.24-7.30 (m, 1H), 6.99-7.02 (m, 1H), 6.93 (t, J=1.8 Hz, 1H), 6.72-6.76 (m, 1H), 3.95-4.02 (m, 2H), 3.94 (s, 3H), 3.32-3.40 (m, 2H), 3.17 (d, J=6.9 Hz, 2H), 3.13 (s, 1H), 2.58 (s, 3H), 1.69-1.77 (m, 4H), 0.91-0.96 (m, 3H).

### Methyl 4′-(2,4-bis(benzyloxy)-5-isopropylbenzamido)-5-(ethyl(tetrahydro-2H-pyran-4-yl)amino)-4-methyl-[1,1′-biphenyl]-3-carboxylate (20)

In a 100 ml RBF, the corresponding intermediate 18 (3.2 gm, 0.0086 mol), 2,4-bis(benzyloxy)-5-isopropylbenzoic acid (3.27 gm, 0.0086 mol), EDC.HCl (3.3 gm, 0.017 mol), and HOBT (1.7 gm, 0.013 mol) were dissolved in 20 ml of DMF and DIPEA (2.7 gm, 0.022 mol) and were added dropwise to the reaction mixture. The reaction mixture was continued for 3 hours and the progress of the reaction was monitored by TLC. After completion, the reaction mixture was diluted with water (100 ml) and the compound was extracted using ethyl acetate (50 ml×3). The combined organic layer was dried over anhydrous magnesium sulfate and concentrated under reduced pressure. The residue was further purified by performing silica gel column chromatography (ethyl acetate:hexane: 1:3) with a yield of 65%. ¹H NMR (300 MHz, CDCl₃): δ 7.72 (s, 1H), 7.21-7.45 (m, 16H), 6.62 (s, 1H), 5.19 (s, 4H), 4.15 (m, 2H), 3.96 (s, 3H), 3.08-3.31 (m, 5H), 3.29 (m, 1H), 2.54 (s, 3H), 1.61-1.67 (m, 4H), 1.26 (d, J=7.2 Hz, 6H), 0.95 (t, J=6.9 Hz, 3H).

### Methyl 3′-(2,4-bis(benzyloxy)-5-isopropylbenzamido)-5-(ethyl(tetrahydro-2H-pyran-4-yl)amino)-4-methyl-[1,1′-biphenyl]-3-carboxylate (21)

Intermediate 21 was synthesized in 70% yield, employing intermediate 19 as the starting material, using the same experimental procedure as that described for the synthesis of compound 20. ¹H NMR (300 MHz, CDCl₃): δ 9.97 (s, 1H), 8.24 (s, 1H), 7.72 (s, 1H), 7.19-7.53 (m, 15H), 6.67 (s, 1H), 5.20 (s, 2H), 5.18 (s, 2H), 3.96-4.15 (m, 5H), 3.33-3.41 (m, 3H), 3.14 (s, 3H), 2.59 (s, 3H), 1.72-1.76 (m, 4H), 1.27-1.32 (m, 6H), 0.91-0.96 (m, 3H).

### 4′-(2,4-bis(benzyloxy)-5-isopropylbenzamido)-5-(ethyl(tetrahydro-2H-pyran-4-yl)amino)-4-methyl-[1,1′-biphenyl]-3-carboxylic acid (22)

Intermediate 20 (1 gm, 0.0014 mol) was dissolved in 20 ml of dioxane, and lithium hydroxide (0.29 gm, 0.0068 eq) was added slowly to the reaction mixture. The reaction was continued for 3 hours and progress of the reaction was monitored by TLC. After completion of the reaction, pH was adjusted to 5 using 3N HCl, and the resulting precipitates were filtered, washed and dried. The resulting acid was carried to the next step without further purification with a yield of 80%.

### 3′-(2,4-bis(benzyloxy)-5-isopropylbenzamido)-5-(ethyl(tetrahydro-2H-pyran-4-yl)amino)-4-methyl-[1,1′-biphenyl]-3-carboxylic acid (23)

Intermediate 23 was synthesized in 91% yield from starting material 21 in a manner similar to that described for the synthesis of intermediate 22. The resulting acid was carried to the next step without further purification.

### 4′-(2,4-bis(benzyloxy)-5-isopropylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (24)

Intermediate 22 (1 gm, 0.0014 mol), 3-(aminomethyl)-4,6-dimethylpyridin-2(1H)-one (0.21 gm, 0.0014 mol), EDC.HCl (0.53 gm, 0.0028 mol) and HOBT (0.28 gm, 0.0021 mol) were dissolved in 10 ml of DMF, and DIPEA (0.45 gm, 0.0035 mol) was added dropwise to the reaction mixture. The reaction mixture was continued for 3 hours and the progress of the reaction was monitored by TLC. After completion, the reaction mixture was diluted with water (100 ml) and the compound was extracted using ethyl acetate (50 ml×3). The combined organic layer was dried over anhydrous magnesium sulfate and concentrated under reduced pressure. The residue was further purified by performing silica gel column chromatography (ethyl acetate:hexane: 1:2) with a yield of 74%. ¹H NMR (300 MHz, CD₃OD): δ 7.79 (s, 1H), 7.09-7.38 (m, 16H), 6.62 (s, 1H), 6.11 (s, 1H), 5.19 (s, 4H), 4.49 (s, 2H), 4.15 (m, 2H), 3.08-3.31 (m, 5H), 3.29 (s, 1H), 2.53 (s, 3H), 2.33 (s, 3H), 2.28 (s, 3H), 1.61-1.67 (m, 4H), 1.26 (d, J=7.2 Hz, 6H), 0.95 (t, J=6.9 Hz, 3H).

### 3′-(2,4-bis(benzyloxy)-5-isopropylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (25)

Intermediate 25 was synthesized in 82% yield from starting material 23 in a manner similar to that described for the synthesis of intermediate 23. ¹H NMR (300 MHz, CD₃OD): δ 7.98 (s, 1H), 7.79 (s, 1H), 7.34-7.65 (m, 15H), 6.54 (s, 1H), 6.17 (s, 1H), 5.11 (s, 2H), 5.07 (s, 2H), 4.66 (s, 2H), 3.95 (bs, 2H), 3.12-3.33 (m, 6H), 2.51 (s, 3H), 2.35 (s, 3H), 2.26 (s, 3H), 1.59-1.76 (m, 4H), 1.29 (d, J=6.6 Hz, 6H), 0.97 (t, J=7.2 Hz, 3H).

### 4′-(2,4-dihydroxy-5-isopropylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (1)

In a 100 ml RBF, intermediate 24 (0.5 gm, 0.0006 mol) was dissolved in 20 ml of methanol and catalytic Pd/C was added. The reaction mixture was continued at room temperature for 4 hours under hydrogen gas conditions and the progress of the reaction was monitored by using TLC. After completion of the reaction, the mixture was diluted with methanol and passed through celite. The solvent was evaporated in-vacuo and the residue was purified by silica gel column chromatography (ethyl acetate:hexane: 1:1) with a yield of 50%. HPLC purity: 98.73%; mp: 166-168 °C; ¹H NMR (600 MHz, CD₃OD): δ 7.82 (s, 1H), 7.56 (d, J=8.4 Hz, 2H), 7.47 (d, J=9 Hz, 2H), 7.41 (d, J=1.2 Hz, 1H), 7.23 (d, J=1.8 Hz, 1H), 6.34 (s, 1H), 6.10 (s, 1H), 4.49 (s, 2H), 3.90 (d, J=10.8 Hz, 2H), 3.45-3.50 (m, 1H), 3.32-3.62 (m, 2H), 3.19-3.24 (m, 1H), 3.10-3.14 (m, 2H), 2.42 (s, 3H), 2.30 (s, 3H), 2.25 (s, 3H), 1.73 (d, J=12 Hz, 2H), 1.58-1.65 (m, 2H), 1.26 (d, J=7.2 Hz, 6H), 0.87 (t, J=7.2 Hz, 3H). ¹³C NMR (150 MHz, CD₃OD): δ 168.23, 160.13, 159.49, 152.05, 149.21, 143.46, 138.92, 137.98, 137.42, 136.04, 132.84, 127.32, 126.53, 126.09, 123.60, 121.69, 120.65, 109.64, 107.43, 102.24, 71.79, 70.64, 66.85, 60.82, 58.45, 41.78, 35.38, 31.45, 30.47, 26.56, 21.68, 18.87, 18.33, 17.18, 13.78, 12.81, 11.73. HRMS (ESI) for C₃₉H₄₇N₄O₆ (M+H⁺): calcd; 667.3496 found, 667.3474 (M+H⁺).

### 3′-(2,4-dihydroxy-5-isopropylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (2)

Title compound 2 was synthesized in 62% yield from starting material 25 in a manner similar to that described for the synthesis of title compound 1. HPLC purity: 97.25%; mp: 170-172 °C; ¹H NMR (600 MHz, CD₃OD): δ 7.89 (s, 1H), 7.76 (s, 1H), 7.58 (dd, J=8.4 and 1.2 Hz, 1H), 7.48 (d, J=1.8 Hz, 1H), 7.40 (t, J=7.8 Hz, 1H), 7.34 (m, 2H), 6.34 (s, 1H), 6.09 (s, 1H), 4.48 (s, 2H), 3.90 (d, J=10.2 Hz, 2H), 3.33-3.36 (m, 2H), 3.09-3.21 (m, 4H), 2.38 (s, 3H), 2.32 (s, 3H), 2.22 (s, 3H), 1.74 (d, J=11.4 Hz, 2H), 1.59-1.66 (m, 2H), 1.24 (d, J=6.6 Hz, 6H), 0.89 (t, J=7.2 Hz, 3H). ¹³C NMR (150 MHz, CD₃OD): δ 171.65, 168.42, 164.21, 160.16, 159.54, 152.20, 149.33, 143.43, 140.88, 139.05, 138.68, 138.38, 133.19, 128.91, 127.28, 126.12, 123.99, 122.42, 121.32, 120.86, 120.36, 119.68, 109.67, 107.54, 102.31, 66.84, 58.36, 41.80, 35.19, 30.50, 26.61, 21.64, 18.30, 17.16, 13.69, 11.71. HRMS (ESI) for C₃₉H₄₇N₄O₆ [M+H⁺]: caled; 667.3496; found, 667.3489.

### Synthetic Example 2

Scheme 2 shown below is another example of synthesizing the compounds disclosed herein.

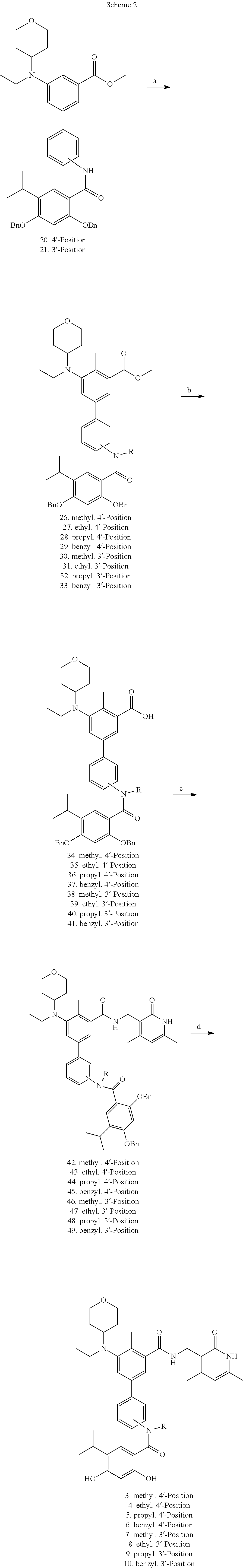

The synthetic route can be used for the target scaffolds 3-10. The adducts 20 and 21 were utilized as versatile starting materials to obtain the N-substituted hybrid templates 3-10. N-alkylation/benzylation of the intermediates 20 and 21 with alkyl iodides and benzyl bromides was afforded by exploiting the catalytic efficiency of NaH as a base at room temperature. The resulting intermediates 26-33 were subjected to lithium hydroxide-assisted ester hydrolysis to obtain the carboxylic acids 34-41 that were subsequently amidated with 3-(aminomethyl)-4,6-dimethylpyridin-2(1H)-one. The amides 42-44 and 46-48 were debenzylated using 10% Pd/C to accomplish the target hybrids 3-5 and 7-9. It is noteworthy to mention that debenzylation of intermediates 45 and 49 was also attempted through palladium-mediated hydrogenation protocol; however, the methodology proved too capricious. It was intriguing to observe that even at room temperature a competition between N-debenzylation and O-debenzylation was observed (not shown in the scheme). Thus, a different tactic to selectively debenzylate the O-benzyl groups was optimized and BCl₃ was used to attain such selectivity. Delightfully, both the palladium-based and the BCl₃-based methodologies led to satisfactory yields of the target compounds.

In particular, the reagents and conditions used in Scheme 2 are as follows: a) alkyl iodide and Benzyl bromide, NaH, DMF, rt, 4 h; b) LiOH(aq), dioxane, rt, 3 h; c) 3-(aminomethyl)-4,6-dimethylpyridin-2(1H)-one, EDC.HCl, HOBT, DIPEA, DMF, rt, 3 h; d) for 42-44 and 46-48, Pd/C, H₂, MeOH, rt, 4 h; for 45 and 49, BCl₃, DCM, rt, 3 h.

### Methyl 4′-(2,4-bis(benzyloxy)-5-isopropyl-N-methylbenzamido)-5-(ethyl(tetrahydro-2H-pyran-4-yl)amino)-4-methyl-[1,1′-biphenyl]-3-carboxylate (26)

In a 100 ml RBF, the intermediate 20 (6 gm, 0.0082 mol) and methyl iodide (3.49 gm, 0.025 eq) were dissolved in 30 ml of DMF and portion-wise addition of sodium hydride (0.39 gm, 0.016 eq) was done. The reaction was stirred at room temperature under nitrogen conditions for 4 hours and the progress of the reaction was monitored using TLC. After completion, the reaction mixture was diluted with water (100 ml) and the compound was extracted using ethyl acetate (50 ml×3). The combined organic layer was dried over anhydrous magnesium sulfate and concentrated under reduced pressure. The residue was further purified by performing silica gel column chromatography (ethyl acetate:hexane: 1:1) with a yield of 51%. ¹H NMR (300 MHz, CD₃OD): δ 7.75 (s, 1H), 7.29-7.38 (m, 16H), 6.29 (s, 1H), 5.19 (s, 2H), 5.16 (s, 2H), 4.18 (m, 2H), 3.91 (s, 3H), 3.41 (s, 3H), 3.29 (s, 1H), 3.11-3.21 (m, 5H), 2.51 (s, 3H), 1.58-1.63 (m, 4H), 1.29 (d, J=7.2 Hz, 6H), 0.92 (t, J=6.9 Hz, 3H).

### Methyl 4′-(2,4-bis(benzyloxy)-N-ethyl-5-isopropylbenzamido)-5-(ethyl(tetrahydro-2H-pyran-4-yl)amino)-4-methyl-[1,1′-biphenyl]-3-carboxylate (27)

Intermediate 27 was synthesized in 69% yield from starting material 20 using ethyl iodide, employing the experimental procedure in a manner similar to that described for compound 26. ¹H NMR (300 MHz, CD₃OD): δ 7.68 (s, 1H), 7.32-7.39 (m, 16H), 6.59 (s, 1H), 5.11 (s, 2H), 5.09 (s, 2H), 3.98 (m, 4H), 3.87 (s, 3H), 3.11-3.27 (m, 6H), 2.46 (s, 3H), 1.58-1.63 (m, 4H), 1.29 (d, J=7.2 Hz, 6H), 1.21 (t, J=7.2 Hz, 3H), 0.89 (t, J=6.9 Hz, 3H).

### Methyl 4′-(2,4-bis(benzyloxy)-5-isopropyl-N-propylbenzamido)-5-(ethyl(tetrahydro-2H-pyran-4-yl)amino)-4-methyl-[1,1′-biphenyl]-3-carboxylate (28)

Intermediate 28 was synthesized in 58% yield from starting material 20 using propyl iodide in a manner similar to that described for compound 26. ¹H NMR (300 MHz, CD₃OD): δ 7.59 (s, 1H), 7.32-7.39 (m, 16H), 6.64 (s, 1H), 5.14 (s, 2H), 5.11 (s, 2H), 3.88-3.92 (m, 4H), 3.89 (s, 3H), 3.17-3.22 (m, 6H), 2.41 (s, 3H), 1.47-1.62 (m, 6H), 1.29 (d, J=7.2 Hz, 6H), 1.18 (t, J=7.2 Hz, 3H), 0.94 (t, J=6.9 Hz, 3H).

### Methyl′-(N-benzyl-2,4-bis(benzyloxy)-5-isopropylbenzamido)-5-(ethyl(tetrahydro-2H-pyran-4-yl)amino)-4-methyl-[1,1′-biphenyl]-3-carboxylate (29)

Intermediate 29 was synthesized in 65% yield from starting material 20 using benzyl iodide in a manner similar to that described for compound 26. ¹H NMR (300 MHz, CD₃OD): δ 7.52 (s, 1H), 7.24-7.41 (m, 21H), 6.62 (s, 1H), 5.11 (s, 2H), 5.08 (s, 2H), 5.04 (s, 2H), 3.93 (m, 2H), 3.91 (s, 3H), 3.11-3.20 (m, 6H), 2.39 (s, 3H), 1.43-1.57 (m, 4H), 1.29 (d, J=7.2 Hz, 6H), 0.94 (t, J=6.9 Hz, 3H).

### Methyl 3′-(2,4-bis(benzyloxy)-5-isopropyl-N-methylbenzamido)-5-(ethyl(tetrahydro-2H-pyran-4-yl)amino)-4-methyl-[1,1′-biphenyl]-3-carboxylate (30)

Intermediate 30 was synthesized in 70% yield from starting material 21 in a manner similar to that described for the synthesis of compound 26. ¹H NMR (300 MHz, CDCl₃): δ 7.46 (s, 1H), 7.13-7.28 (m, 14H), 6.90-6.96 (m, 2H), 6.20 (s, 1H), 4.83 (s, 2H), 4.79 (s, 2H), 3.85 (m, 2H), 3.81 (s, 3H), 3.38 (s, 3H), 2.88-3.20 (m, 6H), 2.42 (s, 3H), 1.55-1.67 (m, 4H), 0.95 (d, J=5.7 Hz, 6H), 0.88 (t, J=3.6 Hz, 3H).

### Methyl 3′-(2,4-bis(benzyloxy)-N-ethyl-5-isopropylbenzamido)-5-(ethyl(tetrahydro-2H-pyran-4-yl)amino)-4-methyl-[1,1′-biphenyl]-3-carboxylate (31)

Intermediate 31 was synthesized in 74% yield from starting material 21 using ethyl iodide in a manner similar to that described for the synthesis of compound 26. ¹H NMR (300 MHz, CDCl₃): δ 7.49 (s, 1H), 7.17-7.22 (m, 14H), 6.93-6.95 (m, 2H), 6.16 (s, 1H), 4.87 (s, 2H), 4.71 (s, 2H), 3.98 (m, 4H), 3.91 (s, 3H), 3.15-3.32 (m, 6H), 2.42 (s, 3H), 1.51-1.68 (m, 4H), 1.32 (d, J=7.2 Hz, 6H), 1.34 (t, J=7.2 Hz, 3H), 0.91 (t, J=6.9 Hz, 3H).

### Methyl 3′-(2,4-bis(benzyloxy)-5-isopropyl-N-propylbenzamido)-5-(ethyl(tetrahydro-2H-pyran-4-yl)amino)-4-methyl-[1,1′-biphenyl]-3-carboxylate (32)

Intermediate 32 was synthesized in 62% yield from starting material 21 using propyl iodide in a manner similar to that described for the synthesis of compound 26. ¹H NMR (300 MHz, CDCl₃): δ 7.44 (s, 1H), 7.19-7.25 (m, 14H), 6.97-6.99 (m, 2H), 6.21 (s, 1H), 4.97 (s, 2H), 4.95 (s, 2H), 3.95-3.97 (m, 4H), 3.89 (s, 3H), 3.21-3.29 (m, 6H), 2.49 (s, 3H), 1.52-1.67 (m, 6H), 1.31 (d, J=7.2 Hz, 6H), 1.21 (t, J=7.2 Hz, 3H), 0.97 (t, J=6.9 Hz, 3H).

### Methyl 3′-(N-benzyl-2,4-bis(benzyloxy)-5-isopropylbenzamido)-5-(ethyl(tetrahydro-2H-pyran-4-yl)amino)-4-methyl-[1,1′-biphenyl]-3-carboxylate (33)

Intermediate 33 was synthesized in 69% yield from starting material 21 using benzyl bromide in a manner similar to that described for compound 26. ¹H NMR (300 MHz, CDCl₃): δ 7.42 (s, 1H), 7.21-7.28 (m, 19H), 6.98-7.01 (m, 2H), 6.25 (s, 1H), 5.01 (s, 2H), 4.98 (s, 2H), 4.95 (s, 2H), 3.99 (m, 2H), 3.89 (s, 3H), 3.18-3.29 (m, 6H), 2.39 (s, 3H), 1.49-1.51 (m, 4H), 1.32 (d, J=7.2 Hz, 6H), 0.98 (t, J=6.9 Hz, 3H).

### 4′-(2,4-bis(benzyloxy)-5-isopropyl-N-methylbenzamido)-5-(ethyl(tetrahydro-2H-pyran-4-yl)amino)-4-methyl-[1,1′-biphenyl]-3-carboxylic acid (34)

Intermediate 26 (6 gm, 0.0081 mol) was dissolved in 30 ml of dioxane, and lithium hydroxide solution (1.66 gm, 0.04 mol) was added dropwise in the reaction mixture. The reaction was continued for 3 hours at room temperature and the progress of the reaction was monitored using TLC. After completion of the reaction, pH was adjusted to 5 using 3N HCl, and the resulting precipitates were filtered, washed and dried. The resulting acid was carried to the next step without further purification with a yield of 79%.

### 4′-(2,4-bis(benzyloxy)-N-ethyl-5-isopropylbenzamido)-5-(ethyl(tetrahydro-2H-pyran-4-yl)amino)-4-methyl-[1,1′-biphenyl]-3-carboxylic acid (35)

Intermediate 35 was synthesized in 56% yield from starting material 27 in a manner similar to that described for compound 34. The carboxylic acid was used for the next step without purification.

### 4′-(2,4-bis(benzyloxy)-5-isopropyl-N-propylbenzamido)-5-(ethyl(tetrahydro-2H-pyran-4-yl)amino)-4-methyl-[1,1′-biphenyl]-3-carboxylic acid (36)

Intermediate 36 was synthesized in 49% yield from starting material 28 in a manner similar to that described for compound 34. The carboxylic acid was used for the next step without purification.

### 4′-(N-benzyl-2,4-bis(benzyloxy)-5-isopropylbenzamido)-5-(ethyl(tetrahydro-2H-pyran-4-yl)amino)-4-methyl-[1,1′-biphenyl]-3-carboxylic acid (37)

Intermediate 37 was synthesized in 57% yield from starting material 29 in a manner similar to that described for compound 34. The carboxylic acid was used for the next step without purification.

### 3′-(2,4-bis(benzyloxy)-5-isopropyl-N-methylbenzamido)-5-(ethyl(tetrahydro-2H-pyran-4-yl)amino)-4-methyl-[1,1′-biphenyl]-3-carboxylic acid (38)

Intermediate 38 was synthesized in 48% yield from starting material 30 in a manner similar to that described for compound 34. The carboxylic acid was used for the next step without purification.

### 3′-(2,4-bis(benzyloxy)-N-ethyl-5-isopropylbenzamido)-5-(ethyl(tetrahydro-2H-pyran-4-yl)amino)-4-methyl-[1,1′-biphenyl]-3-carboxylic acid (39)

Intermediate 39 was synthesized in 71% yield from starting material 31 in a manner similar to that described for compound 34. The carboxylic acid was used for the next step without purification.

### 3′-(2,4-bis(benzyloxy)-5-isopropyl-N-propylbenzamido)-5-(ethyl(tetrahydro-2H-pyran-4-yl)amino)-4-methyl-[1,1′-biphenyl]-3-carboxylic acid (40)

Intermediate 40 was synthesized in 43% yield from starting material 32 in a manner similar to that for compound 34. The carboxylic acid was used for the next step without purification.

### 3′-(N-benzyl-2,4-bis(benzyloxy)-5-isopropylbenzamido)-5-(ethyl(tetrahydro-2H-pyran-4-yl)amino)-4-methyl-[1,1′-biphenyl]-3-carboxylic acid (41)

Intermediate 41 was synthesized in 55% yield from starting material 33 in a manner similar to that described for compound 34. The carboxylic acid was used for the next step without purification

### 4′-(2,4-bis(benzyloxy)-5-isopropyl-N-methylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (42)

In a 100 ml RBF, the intermediate 34 (5.3 gm, 0.0073 mol), 3-(aminomethyl)-4,6-dimethylpyridin-2(1H)-one (1.37 gm, 0.0073 mol), EDC.HCl (2.79 gm, 0.0145 mol) and HOBT (1.48 gm, 0.011 mol) were dissolved in 30 ml of DMF, and DIPEA (2.35 gm, 0.018 mol) was added dropwise in the reaction mixture. The reaction was continued for 3 hours and the progress was monitored by TLC. After completion, the reaction mixture was diluted with water (100 ml) and the compound was extracted using ethyl acetate (50 ml×3). The combined organic layer was dried over anhydrous magnesium sulfate and concentrated under reduced pressure. The residue was further purified by performing silica gel column chromatography (ethyl acetate:Hexane: 1:1) with a yield of 62%. ¹H NMR (300 MHz, CD₃OD): δ 7.73 (s, 1H), 7.11-7.29 (m, 16H), 6.66 (s, 1H), 6.15 (s, 1H), 5.23 (s, 2H), 5.15 (s. 2H), 4.53 (s, 2H), 3.99 (q, J=6.6 Hz, 2H), 3.31 (m, 1H), 3.11-3.21 (m, 5H), 2.49 (s, 3H), 2.31 (s, 3H), 2.22 (s, 3H), 1.66-1.71 (m, 4H), 1.23 (d, J=7.2 Hz, 6H), 0.91 (t, J=6.9 Hz, 3H).

### 4′-(2,4-bis(benzyloxy)-N-ethyl-5-isopropylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (43)

The intermediate 43 was obtained in 56% yield from starting material 35 in a manner similar to that described for the synthesis of compound 42. ¹H NMR (300 MHz, CD₃OD): δ 7.69 (s, 1H), 7.22-7.38 (m, 16H), 6.51 (s, 1H), 6.11 (s, 1H), 5.15 (s, 2H), 5.11 (s, 2H), 4.51 (bs, 2H) 3.91-3.95 (m, 4H), 3.29 (s, 3H), 3.15-3.25 (m, 6H), 2.49 (s, 3H), 2.32 (s, 3H), 2.24 (s, 3H), 1.63-1.69 (m, 4H), 1.31 (d, J=7.2 Hz, 6H), 1.25 (t, J=7.2 Hz, 3H), 0.92 (t, J=6.9 Hz, 3H).

### 4′-(2,4-bis(benzyloxy)-5-isopropyl-N-propylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (44)

The intermediate 44 was obtained in 58% yield from starting material 36 in a manner similar to that described for the synthesis of compound 42. ¹H NMR (300 MHz, CD₃OD): δ 7.61 (s, 1H), 7.32-7.39 (m, 16H), 6.68 (s, 1H), 6.12 (s, 1H), 5.17 (s, 2H), 5.18 (s, 2H), 4.43 (bs, 2H), 3.92-3.95 (m, 4H), 3.21-3.29 (m, 6H), 2.49 (s, 3H), 2.31 (s, 3H), 2.25 (s, 3H), 1.45-1.67 (m, 6H), 1.31 (d, J=7.2 Hz, 6H), 1.16 (t, J=7.2 Hz, 3H), 0.92 (t, J=6.9 Hz, 3H).

### 4′-(N-benzyl-2,4-bis(benzyloxy)-5-isopropylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (45)

The intermediate 45 was obtained in 60% yield from starting material 37 in a manner similar to that described for the synthesis of compound 42. ¹H NMR (300 MHz, CD₃OD): δ 7.56 (s, 1H), 7.21-7.38 (m, 22H), 6.64 (s, 1H), 5.13 (s, 2H), 5.09 (s, 2H), 5.08 (s, 2H), 4.51 (bs, 2H), 3.92 (m, 2H), 3.15-3.25 (m, 6H), 2.49 (s, 3H), 2.29 (s, 3H), 2.21 (s, 3H), 1.48-1.54 (m, 4H), 1.25 (d, J=7.2 Hz, 6H), 0.91 (t, J=6.9 Hz, 3H).

### 3′-(2,4-bis(benzyloxy)-5-isopropyl-N-methylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (46)

The intermediate 46 was obtained in 63% yield from starting material 38 in a manner similar to that described for the synthesis of compound 42. ¹H NMR (300 MHz, DMSO-d₆) δ 11.46 (s, 1H), 8.20 (s, 1H), 7.33-7.45 (m, 13H), 7.21 (s, 1H), 7.09 (d, J=7.2 Hz, 1H), 7.04 (s, 1H), 6.88 (s, 1H), 6.77 (s, 1H), 5.85 (s, 1H), 5.11 (s, 2H), 5.05 (s, 2H), 4.33 (d, J=4.5 Hz, 2H), 3.81 (d, J=10.5 Hz, 2H), 3.22 (t, J=10.8 Hz, 2H), 3.04 (m, 4H), 2.22 (d, J=4.2 Hz, 5H), 2.10 (s, 3H), 2.07 (s, 3H), 1.62 (d, J=11.1 Hz, 2H), 1.52 (d, J=11.1 Hz, 2H), 1.25 (s, 2H), 0.93 (s, 5H), 0.79 (t, J=6.6 Hz, 3H),

### 3′-(2,4-bis(benzyloxy)-N-ethyl-5-isopropylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (47)

The intermediate 47 was obtained in 36% yield from starting material 39 in a manner similar to that described for the synthesis of compound 42. ¹H NMR (300 MHz, CD₃OD): δ 7.51 (s, 1H), 7.19-7.23 (m, 14H), 6.94 (m, 2H), 6.16 (s, 1H), 6.09 (s, 1H), 5.03 (s, 2H), 5.01 (s, 2H), 4.68 (s, 2H), 3.95-3.98 (m, 4H), 3.89 (s, 3H), 3.19-3.21 (m, 6H), 2.49 (s, 3H), 2.33 (s, 3H), 2.21 (s, 3H), 1.58-1.71 (m, 4H), 1.38 (d, J=7.2 Hz, 6H), 1.31 (t, J=7.2 Hz, 3H), 0.89 (t, J=6.9 Hz, 3H).

### 3′-(2,4-bis(benzyloxy)-5-isopropyl-N-propylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (48)

Intermediate 48 was synthesized in 62% yield from starting material 40 in a manner similar to that described for compound 42. ¹H NMR (300 MHz, CDCl₃): δ 7.49 (s, 1H), 7.21-7.27 (m, 14H), 6.94-6.97 (m, 2H), 6.11 (s, 1H), 4.97 (s, 2H), 4.95 (s, 2H), 4.51 (d, J=5.4 Hz, 2H), 3.96-3.99 (m, 4H), 3.91 (s, 3H), 3.25-3.31 (m, 6H), 2.51 (s, 3H), 2.31 (s, 3H), 2.21 (s, 3H), 1.55-1.72 (m, 6H), 1.23 (d, J=7.2 Hz, 6H), 1.21 (t, J=7.2 Hz, 3H), 0.97 (t, J=6.9 Hz, 3H).

### 3′-(N-benzyl-2,4-bis(benzyloxy)-5-isopropylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (49)

Intermediate 49 was synthesized in 38% yield from starting material 41 in a manner similar to that described for compound 42. ¹H NMR (300 MHz, CDCl₃): δ 7.51 (s, 1H), 7.28-7.31 (m, 19H), 7.03 (m, 2H), 6.27 (s, 1H), 5.02 (s, 2H), 4.99 (s, 2H), 4.97 (s, 2H), 4.44 (d, J=5.4 Hz, 2H), 4.01 (s, 2H), 3.92 (s, 3H), 3.21-3.25 (m, 6H), 2.41 (s, 3H), 2.31 (s, 3H), 2.25 (s, 3H), 1.52-1.56 (m, 4H), 1.29 (d, J=7.2 Hz, 6H), 0.99 (t, J=6.9 Hz, 3H).

### 4′-(2,4-dihydroxy-5-isopropyl-N-methylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (3)

In a 100 ml RBF, intermediate 42 (0.6 gm, 0.0007 mol) was dissolved in 20 ml of methanol and catalytic Pd/C was added. The reaction was continued at room temperature for 4 hours, under hydrogen gas conditions and the progress of the reaction was monitored by using TLC. After completion, the mixture was diluted with methanol and passed through celite. The solvent was evaporated in-vacuo and the residue was purified by silica gel column chromatography (ethyl acetate:hexane: 1:1) with yields of 47%. HPLC purity: 96.16%; mp: 184-186 °C; ¹H NMR (600 MHz, DMSO-d₆): δ 11.43 (s, 1H), 10.69 (s, 1H), 9.72 (s, 1H), 8.15 (t, J=5.4 Hz, 1H), 7.54 (d, J=8.4 Hz, 2H), 7.27 (d, J=1.2 Hz, 1H), 7.23 (d, J=8.4 Hz, 2H), 7.14 (d, J=1.8 Hz, 1H), 6.51 (s, 1H), 6.18 (s, 1H), 5.82 (s, 1H), 4.25 (d, J=5.4 Hz, 2H), 3.79 (d, J=10.2 Hz, 2H), 3.33 (s, 3H), 3.21 (t, J=10.8 Hz, 2H), 3.01-3.04 (m, 2H), 2.95-3.00 (m, 1H), 2.77-2.82 (m, 1H), 2.21 (s, 3H), 2.17 (s, 3H), 2.07 (s, 3H), 1.62 (d, J=12.6 Hz, 2H), 1.44-1.51 (m, 2H), 0.78 (t, J=7.2 Hz, 3H), 0.68 (d, J=6.6 Hz, 6H). ¹³C NMR (150 MHz, DMSO-d₆): δ 170.76, 170.67, 169.39, 163.44, 158.22, 157.97, 149.94, 149.27, 144.84, 143.18, 140.06, 138.55, 136.85, 133.30, 128.22, 127.84, 127.43, 125.09, 123.47, 122.05, 121.21, 109.76, 107.78, 102.72, 66.75, 60.18, 58.14, 41.56, 38.47, 35.29, 30.69, 25.46, 22.64, 21.19, 19.36, 18.62, 14.96, 14.52, 12.98. HRMS (ESI) for C₄₀H₄₉N₄O₆ [M+H⁺]: calcd; 681.3652 found, 681.3625.

### N-((4,6-dimethyl-2-oxo-1,2-dihydropyridin-3-yl)methyl)-5-(ethyl(tetrahydro-2H-pyran-4-yl)amino)-4′-(N-ethyl-2,4-dihydroxy-5-isopropylbenzamido)-4-methyl-[1,1′-biphenyl]-3-carboxamide (4)

The title compound 4 was synthesized in 42% yield from starting material 43 in a manner similar to that described for compound 3. HPLC purity: 98.38%; mp: 191-193 °C; ¹H NMR (600 MHz, DMSO-d₆): δ 11.44 (s, 1H), 10.89 (s, 1H), 9.74 (s, 1H), 8.15 (t, J=4.8 Hz, 1H), 7.56 (d, J=8.4 Hz, 2H), 7.28 (d, J=1.2 Hz, 1H), 7.21 (d, J=8.4 Hz, 2H), 7.15 (d, J=1.2 Hz, 1H), 6.48 (s, 1H), 6.18 (s, 1H), 5.82 (s, 1H), 4.26 (d, J=4.8 Hz, 2H), 3.81-3.85 (m, 2H), 3.79 (d, J=10.2 Hz, 2H), 3.21 (t, J=11.4 Hz, 2H), 3.01-3.05 (m, 2H), 2.77 (t, J=6.6 Hz, 1H), 2.21 (s, 3H), 2.17 (s, 3H), 2.07 (s, 3H), 1.56 (d, J=12 Hz, 2H), 1.44-1.51 (m, 2H), 1.09 (t, J=7.2 Hz, 4H), 0.78 (t, J=7.2 Hz, 3H), 0.66 (d, J=6.6 Hz, 6H). ¹³C NMR (150 MHz, DMSO-d₆): δ 170.39, 169.39, 163.45, 158.06, 149.96, 149.28, 143.19, 143.08, 140.05, 138.82, 136.83, 133.36, 128.40, 128.26, 127.93, 125.03, 123.51, 122.05, 121.26, 107.80, 102.71, 66.75, 58.14, 45.42, 41.58, 35.29, 30.70, 25.43, 23.16, 22.62, 19.36, 18.62, 14.97, 13.05, 12.97. HRMS (ESI) for C₄₁H₅₁N₄O₆ [M+H⁺]: caled; 695.3809; found, 695.3757 (M+H⁺).

### 4′-(2,4-dihydroxy-5-isopropyl-N-propylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (5)

The title compound 5 was synthesized in 55% yield from starting material 44 in a manner similar to that described for compound 3. HPLC purity: 95.75%; mp: 155-157 °C; ¹H NMR (600 MHz, CD₃OD): δ 7.54 (d, J=8.4 Hz, 2H), 7.38 (s, 1H), 7.26 (s, 1H), 7.21 (d, J=7.8 Hz, 2H), 6.55 (s, 1H), 6.19 (s, 1H), 6.10 (s, 1H), 4.48 (s, 2H), 3.87-3.92 (m, 4H), 3.35 (t, J=11.4 Hz, 2H), 3.11-3.14 (m, 2H), 3.06-3.09 (m, 1H), 2.83-2.88 (m, 1H), 2.38 (s, 3H), 2.31 (s, 3H), 2.23 (s, 3H), 1.73 (d, J=12 Hz, 2H), 1.60-1.68 (m, 4H), 0.94 (t, J=7.8 Hz, 3H), 0.87 (t, J=7.2 Hz, 3H), 0.70 (d, J=6.6 Hz, 6H). ¹³C NMR (150 MHz, CD₃OD): δ 171.53, 164.21, 159.03, 158.12, 152.14, 149.29, 143.44, 143.41, 139.17, 139.08, 137.66, 133.27, 128.17, 127.71, 127.55, 125.36, 124.02, 121.33, 120.73, 109.63, 108.62, 101.89, 66.83, 58.27, 51.88, 41.70, 35.16, 30.42, 25.35, 21.46, 20.41, 18.27, 17.14, 13.62, 11.59, 10.22. HRMS (ESI) for C₄₂H₅₃N₄O₆ [M+H⁺]: calcd; 709.3965; found, 709.3976.

### 4′-(N-benzyl-2,4-dihydroxy-5-isopropylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (6)

Intermediate 45 (0.5 gm, 0.0006 mol) was dissolved in 20 ml of DCM, and 2.3 ml of 1M BCl₃ solution in DCM (0.27 gm, 0.0023 mol) was added dropwise to the solution. The reaction mixture was stirred for 2 hours and the progress of the reaction was monitored. After completion (TLC), the reaction mixture was quenched with water and extraction was done with DCM (50 ml×3). The separated organic layer was evaporated using a rotary evaporator. The residue was purified by performing silica gel column chromatography (ethylacetate:Hexane: 2:1) to give the target compound 6 in 51% yield. HPLC purity: 97.24%; mp: 135-137 °C; ¹H NMR (600 MHz, DMSO-d₆): δ 11.41 (s, 1H), 10.54 (s, 1H), 8.10 (t, J=4.8 Hz, 1H), 7.46 (d, J=8.4 Hz, 2H), 7.27-7.30 (m, 4H), 7.24 (s, 2H), 7.19 (t, J=7.2 Hz, 1H), 7.13 (d, J=8.4.2 Hz, 2H), 7.10 (d, J=1.8 Hz, 1H), 6.57 (s, 1H), 6.20 (s, 1H), 5.81 (s, 1H), 5.07 (s, 2H), 4.24 (d, J=4.8 Hz, 2H), 3.77 (d, J=10.8 Hz, 2H), 3.34 (m, 1H), 3.19 (t, J=10.8 Hz, 2H), 2.98-3.02 (m, 2H), 2.77-2.82 (m, 1H), 2.19 (s, 3H), 2.15 (s, 3H), 2.07 (s, 3H), 1.59 (d, J=11.4 Hz, 2H), 1.42-1.46 (m, 2H), 0.75 (t, J=7.8 Hz, 3H), 0.69 (d, J=6.6 Hz, 6H). ¹³C NMR (150 MHz, DMSO-d₆): δ 170.71, 169.36, 163.42, 157.97, 149.92, 149.27, 143.17, 139.99, 138.35, 137.99, 136.64, 133.33, 128.79, 128.16, 127.95, 127.53, 125.20, 123.39, 122.04, 121.17, 107.75, 102.74, 72.44, 70.41, 66.73, 60.69, 58.11, 53.25, 35.28, 31.79, 30.71, 29.45, 25.52, 22.88, 22.65, 22.52, 19.36, 19.26, 18.62, 14.96, 14.23, 12.97. HRMS (ESI) for C₄₆H₅₃N₄O₆ [M+H⁺]: caled; 757.3965; found, 757.3932.

### 3′-(2,4-dihydroxy-5-isopropyl-N-methylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (7)

The title compound 7 was synthesized in 48% yield from starting material 46 in a manner similar to that described for compound 3. HPLC purity: 95.14%; mp: 161-163 °C; ¹H NMR (600 MHz, CD₃OD): δ 7.46 (t, J=7.8 Hz, 1H), 7.39 (d, J=7.8 Hz, 1H), 7.29 (d, J=6.6 Hz, 1H), 7.16 (d, J=1.8 Hz, 1H), 7.11 (t, J=1.8 Hz, 1H), 6.98 (d, J=1.8 Hz, 1H), 6.57 (s, 1H), 6.27 (s, 1H), 6.10 (s, 1H), 4.47 (s, 2H), 3.90 (d, J=10.8 Hz, 2H), 3.47 (s, 3H), 3.33-3.37 (m, 2H), 3.02-3.06 (m, 2H), 2.37 (s, 3H), 2.28 (s, 3H), 2.23 (s, 3H), 1.68 (d, J=11.4 Hz, 2H), 1.57-1.62 (m, 2H), 1.29 (d, J=5.4 Hz, 2H) 0.82 (t, J=7.2 Hz, 3H), 0.68 (d, J=6.6 Hz, 6H). ¹³C NMR (150 MHz, CD₃OD): δ 171.98, 171.52, 164.21, 158.72, 158.21, 152.23, 149.26, 146.14, 143.47, 141.67, 139.08, 137.76, 133.41, 129.76, 128.31, 125.86, 125.54, 124.67, 124.27, 124.16, 121.26, 120.68, 109.70, 108.59, 101.99, 66.81, 58.22, 41.71, 37.24, 35.20, 30.42, 25.32, 21.50, 18.27, 17.15, 13.58, 11.60. HRMS (ESI) for C₄₀H₄₉N₄O₆ [M+H⁺]: calcd; 681.3652; found, 681.3623.

### N-((4,6-dimethyl-2-oxo-1,2-dihydropyridin-3-yl)methyl)-5-(ethyl(tetrahydro-2H-pyran-4-yl)amino)-3′-(N-ethyl-2,4-dihydroxy-5-isopropylbenzamido)-4-methyl-[1,1′-biphenyl]-3-carboxamide (8)

The title compound 8 was synthesized in 68% yield from starting material 47 in a manner similar to that described for compound 3. HPLC purity: 95.02%; mp: 151-153 °C; ¹H NMR (600 MHz, CD₃OD): δ 7.46 (t, J=7.8 Hz, 1H), 7.40 (d, J=7.8 Hz, 1H), 7.29 (d, J=7.8 Hz, 1H), 7.16 (d, J=1.8 Hz, 1H), 7.08 (s, 1H), 6.98 (s, 1H), 6.57 (s, 1H), 6.26 (s, 1H), 6.10 (s, 1H), 4.47 (s, 2H), 4.00 (s, 2H), 3.91 (d, J=10.8 Hz, 2H), 3.35 (t, J=10.2 Hz, 2H), 3.03-3.06 (m, 2H), 2.97-3.00 (m, 1H), 2.83-2.87 (m, 1H), 2.37 (s, 3H), 2.28 (s, 3H), 2.23 (s, 3H), 1.68 (d, J=11.4 Hz, 2H), 1.56-1.62 (m, 2H), 1.21 (t, J=6.6 Hz, 3H) 0.82 (t, J=7.2 Hz, 3H), 0.68 (d, J=7.2 Hz, 6H). ¹³C NMR (150 MHz, CD₃OD): δ 171.57, 158.90, 158.14, 152.23, 149.28, 144.42, 143.47, 141.70, 139.07, 137.78, 133.43, 129.75, 129.42, 128.25, 126.88, 125.48, 124.83, 124.76, 124.29, 120.72, 109.69, 108.72, 101.95, 66.82, 58.25, 45.05, 41.72, 35.19, 35.12, 31.63, 30.42, 25.32, 22.30, 21.50, 18.26, 17.14, 13.58, 13.00, 11.63, 11.61. HRMS (ESI) for C₄₁H₅₁N₄O₆ [M+H⁺]: calcd; 695.3809; found, 695.3773.

### 3′-(2,4-dihydroxy-5-isopropyl-N-propylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (9)

The title compound 9 was synthesized in 71% yield from starting material 48 in a manner similar to that described for compound 3. HPLC purity: 97.52%; mp: 172-175 °C; ¹H NMR (600 MHz, DMSO-d₆): δ 11.43 (s, 1H), 10.56 (s, 1H), 9.69 (s, 1H), 8.11 (t, J=4.8 Hz, 1H), 7.36-7.40 (m, 2H), 7.26 (s, 1H), 7.16 (d, J=7.2 Hz, 1H), 7.10 (d, J=1.2 Hz, 1H), 7.03 (s, 1H), 6.50 (s, 1H), 6.21 (s, 1H), 5.83 (s, 1H), 4.26 (d, J=4.8 Hz, 2H), 3.79 (t, J=7.8 Hz, 4H), 3.22 (t, J=10.8 Hz, 2H), 2.97-3.01 (m, 2H), 2.89-2.94 (m, 1H), 2.74-2.79 (m, 1H), 2.19 (s, 3H), 2.17 (s, 3H), 2.07 (s, 3H), 1.59 (d, J=11.4 Hz, 2H), 1.46-1.53 (m, 4H), 0.83 (t, J=7.2 Hz, 3H), 0.76 (t, J=7.2 Hz, 3H), 0.65 (d, J=7.2 Hz, 6H). ¹³C NMR (150 MHz, DMSO-d₆): δ 170.55, 169.37, 163.45, 157.80, 150.03, 149.28, 144.65, 141.62, 140.09, 137.05, 133.41, 130.16, 128.00, 126.44, 126.05, 125.22, 125.03, 123.65, 122.02, 121.34, 110.13, 107.85, 102.70, 66.73, 65.34, 58.18, 51.56, 41.56, 35.31, 30.69, 25.41, 22.70, 20.84, 19.37, 18.62, 15.60, 14.95, 13.05, 11.64. HRMS (ESI) for C₄₂H₅₃N₄O₆ [M+H⁺]: caled; 709.3965 found, 709.3956.

### 3′-(N-benzyl-2,4-dihydroxy-5-isopropylbenzamido)-N-((4,6-dimethyl-2-oxo-1,2-dihydropyridin-3-yl)methyl)-5-(ethyl(tetrahydro-2H-pyran-4-yl)amino)-4-methyl-[1,1′-biphenyl]-3-carboxamide (10)

The title compound 10 was synthesized in 53% yield from starting material 49 in a manner similar to that described for compound 6. HPLC purity: 95.26%; mp: 146-148 °C; ¹H NMR (600 MHz, CD₃OD): δ 7.36 (t, J=7.2 Hz, 1H), 7.32 (t, J=9 Hz, 3H), 7.25 (t, J=7.2 Hz, 2H), 7.19 (t, J=7.2 Hz, 2H), 7.05 (d, J=0.6 Hz, 1H), 6.91 (d, J=9 Hz, 2H), 6.64 (s, 1H), 6.27 (s, 1H), 6.10 (s, 1H), 5.16 (s, 2H), 4.47 (s, 2H), 3.90 (d, J=11.4 Hz, 2H), 3.34 (t, J=10.8 Hz, 2H), 3.01-3.04 (m, 2H), 2.94-2.98 (m, 1H), 2.83-2.88 (m, 1H), 2.37 (s, 3H), 2.26 (s, 3H), 2.23 (s, 3H), 1.65 (d, J=12 Hz, 2H), 1.54-1.61 (m, 2H), 0.80 (t, J=7.2 Hz, 3H), 0.68 (d, J=6.6 Hz, 6H). ¹³C NMR (150 MHz, CD₃OD): δ 171.79, 171.50, 164.22, 158.78, 158.25, 152.23, 149.20, 144.41, 143.47, 141.41, 139.04, 137.70, 137.43, 133.34, 129.54, 128.13, 128.10, 128.04, 127.05, 126.85, 125.57, 124.84, 124.81, 124.13, 121.28, 120.65, 109.71, 108.80, 102.00, 66.81, 58.22, 54.64, 52.91, 41.67, 35.17, 30.40, 28.11, 25.36, 21.50, 18.29, 17.15, 13.56, 11.60. HRMS (ESI) for C₄₆H₅₃N₄O₆ [M+H⁺]: calcd; 757.3965 found, 757.3998.

Scheme 3 shown below is another example of synthesizing the compounds disclosed herein.

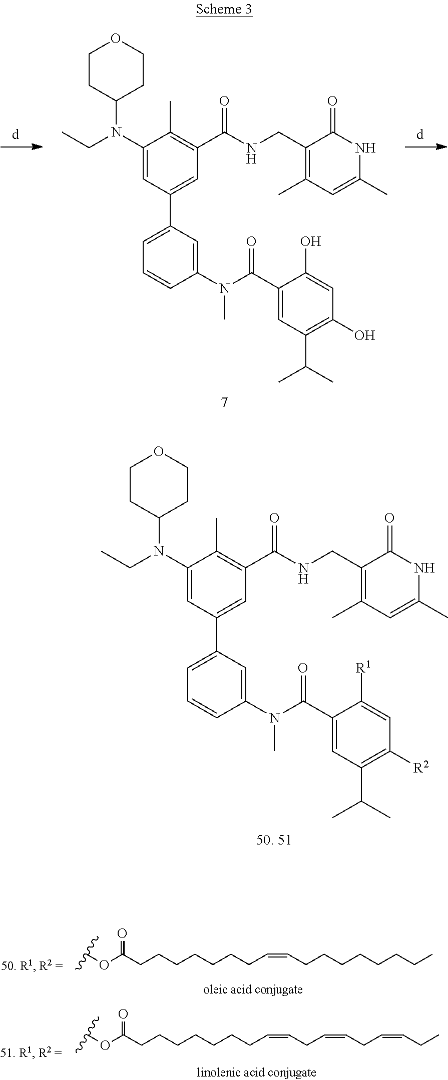

The synthetic route can be used for the target scaffolds 50 and 51. In particular, the reagents and conditions used in Scheme 3 are as follows: d) Pd/C, H2, MeOH, rt, 4 h; e) for 50, oleic acid, DCC, DMAP, DCM, rt, 4 h; for 51, linolenic acid, DCC, DMAP, DCM, rt, 4 h.

### 4-((3′-(((4,6-dimethyl-2-oxo-1,2-dihydropyridin-3-yl)methyl)carbamoyl)-5′-(ethyl(tetrahydro-2H-pyran-4-yl)amino)-4′-methyl-[1,1′-biphenyl]-3-yl)(methyl)carbamoyl)-6-isopropyl-1,3-phenylene dioleate (50)

In a 100 ml RBF, intermediate 7 (0.5 gm, 0.00073 mol), oleic acid (0.4 gm, 0.0014 mol) and DCC (0.75 gm, 0.0036 mol) was dissolved in 20 ml of DCM, and portion-wise, DMAP (0.089 gm, 0.0007 mol) was added in the reaction mixture. The reaction was continued for 4 hours at room temperature under nitrogen conditions and progress of the reaction was monitored using TLC. After completion, the reaction mixture was diluted with water (100 ml) and the compound was extracted using ethyl acetate (50 ml×3). The combined organic layer was dried over anhydrous magnesium sulfate and concentrated under reduced pressure. The residue was further purified by performing silica gel column chromatography (ethyl acetate: Hexane: 1:1) with a yield of 68%. HPLC purity: XX %; mp: 133-135 °C; ¹H NMR (300 MHz, CDCl₃) δ 11.60 (s, 1H), 7.31-7.39 (m, 3H), 7.24 (d, J=6.3 Hz, 2H), 7.10 (d, J=16.5 Hz, 2H), 6.90 (s, 1H), 6.81 (s, 1H), 5.95 (s, 1H), 5.34-5.38 (m, 4H), 4.58 (d, J=5.7 Hz, 2H), 3.98 (d, J=11.1 Hz, 2H), 3.52 (s, 4H), 3.29-3.38 (m, 2H), 3.15 (s, 2H), 2.79 (t, J=5.7 Hz, 1H), 2.59 (d, J=7.5 Hz, 2H), 2.52 (d, J=7.5 Hz, 2H), 2.42 (s, 3H), 2.20 (s, 3H), 2.00-2.04 (m, 7H), 1.92-1.98 (m, 3H), 1.68-1.75 (m, 9H), 1.29-1.33 (m, 40H), 1.09-1.18 (m, 3H), 0.88-0.92 (m, 8H), 0.78 (s, 4H). LRMS (ESI) for C₇₆H₁₁₂N₄O₈Na [M+Na⁺)]: caled; 1231.83; found, 1231.80.

### 4-((3′-(((4,6-dimethyl-2-oxo-1,2-dihydropyridin-3-yl)methyl)carbamoyl)-5′-(ethyl(tetrahydro-2H-pyran-4-yl)amino)-4′-methyl-[1,1′-biphenyl]-3-yl)(methyl)carbamoyl)-6-isopropyl-1,3-phenylene (9Z,9′Z,12Z,12′Z,15Z,15′Z)-bis(octadeca-9,12,15-trienoate) (51)

In a 100 ml RBF, intermediate 7 (0.5 gm, 0.00073 mol), linolenic acid (0.4 gm, 0.0014 mol) and DCC (0.75 gm, 0.0036 mol) were dissolved in 20 ml of DCM, and portion-wise, DMAP (0.089 gm, 0.0007 mol) was added in the reaction mixture. The reaction was continued for 4 hours at room temperature under nitrogen conditions and progress of the reaction was monitored using TLC. After completion, the reaction mixture was diluted with water (100 ml) and the compound was extracted using ethyl acetate (50 ml×3). The combined organic layer was dried over anhydrous magnesium sulfate and concentrated under reduced pressure. The residue was further purified by performing silica gel column chromatography (ethylacetate:Hexane: 1:1) with a yield of 52%. HPLC purity: XX %; mp: 122-124 °C; ¹H NMR (300 MHz, CDCl₃) δ 11.47 (s, 1H), 8.07 (s, 1H), 7.47 (s, 1H), 7.39 (s, 2H), 7.30 (s, 1H), 7.13 (s, 1H), 7.00 (d, J=9.9 Hz, 2H), 6.92 (s, 1H), 5.86 (s, 1H), 5.58 (d, J=3.6 Hz, 1H), 5.32 (s, 11H), 4.30 (s, 2H), 3.84 (d, J=9.9 Hz, 2H), 3.41 (s, 2H), 3.25 (t, J=11.1 Hz, 2H), 3.02 (d, J=5.1 Hz, 2H), 2.93 (s, 1H), 2.77 (s, 7H), 2.55 (s, 2H), 2.22 (t, J=1.8 Hz, 6H), 2.11 (s, 3H), 2.03 (d, J=4.8 Hz, 6H), 1.73 (d, J=12 Hz, 2H), 1.60 (s, 6H), 1.52 (d, J=12.3 Hz, 3H), 1.29 (s, 19H), 1.07 (t, J=10.8 Hz, 2H), 0.90-0.95 (m, 5H), 0.77 (d, J=6.3 Hz, 9H). LRMS (ESI) for C₇₆H₁₀₄N₄O₈Na [M+Na⁺)]: calcd; 1223.77; found, 1223.75.

### Example 3—EZH2 Inhibition

Next, the successful construction of the target adducts (1-10), i.e., profiling of their EZH2 inhibitory potential, was performed. The results of the study are summarized in Table 1 below.

TABLE 1
EZH2 inhibitory activity of the synthesized adducts;
EZH2 inhibitory assays were performed by reaction
biology corporation, Malvern, PA

| | EZH2 (Core Histone) |
|---|---|
| Compound ID: | IC₅₀ (nM) |
| 1 | 6.14 |
| 2 | 2.06 |
| 3 | 7.21 |
| 4 | 7.35 |
| 5 | 21.8 |
| 6 | 11.7 |
| 7 | 6.29 |
| 8 | 36.6 |
| 9 | 40.1 |
| 10 | 641 |
| Tazemetostat | 0.697 |

Careful observation of the results gave us some insights regarding the structure—EZH2 inhibition relationship. Five adducts (1-4, 7) elicited outstanding EZH2 inhibitory potential with IC₅₀ values in the single-digit nanomolar range. The key insight gleaned from correlating the structural features of the adducts with EZH2 inhibitory activity revealed that tetheration of the resorcinol fragment via amide bond at 3′ position was favored over the tetheration at 4′ position, as compound 2 (resorcinol fragment, 3′ position) manifested a striking EZH2 inhibitory activity with an IC₅₀ value of 2.06 nM. Notably, compound 2 was 3-fold more potent in inhibiting EZH2 than its counterpart 1 (resorcinol fragment, 4′ position). To test the tolerance of N-alkylation/benzylation (amide NH), the generated adducts 3-10 were also evaluated. Notably, the impact of N-alkylation/benzylation was quite obviously detrimental for the EZH2 inhibitory potential, and the trend of relatively reduced enzyme inhibition activity was evidenced in regio-counterparts (resorcinol fragment at 4′ as well as 3′ position). A deeper dive into the enzyme inhibitory profile of compound 3-10 perspicuously, clearly ascertains the aforementioned trend as compound 1 outshines its N-alkylated/benzylated derivatives (3-6). Likewise, compound 2 was observed to be stupendously, remarkably more effective in inhibiting the enzymatic catalytic subunit of polycomb repressive complex 2 than its N-alkylated/benzylated derivatives (7-10). The other key structural insights gleaned from the assay were the highest degree of decrease in EZH2 inhibitory activity observed with N-benzylation (N, amide bearing the resorcinol fragments) in both cases (amide-tethered at position 4′ and amide tethered hybrids at position 3′), and the higher magnitude of downward trends witnessed with N-substitution in the hybrid scaffolds flanking the resorcinol fragment at position 3′. Although some potent EZH2 inhibitors were pinpointed through this evaluation study, a relative comparison with the EZH2 inhibitory activity of tazemetostat (I) indicated that even the best four out of the 10 hybrids furnished were endowed with a slightly inferior EZH2 inhibitory profile. It is important to mention that this observation did not prove to be a hindrance in our endeavour, as tazemetostat (I), despite being a magnificently potent EZH2 inhibitor, could not exert cell growth inhibitory potential against the GBM cell lines as per our preliminary screening results, some literature precedents, as well as the comparative analysis report of tazemetostat (I) vs hybrid scaffolds (1-10, Table 6 below). Thus, we anticipated that the balanced concomitant modulation of EZH2 and HSP90 via the furnishing of hybrid scaffolds based on the structural commonalities of pharmacophores of both targets might activate the chemical architecture of tazemetostat to exert anti-GBM effects. In light of this anticipation, our research group was inclined towards the pursuit of identifying a dual EZH2-HSP90 inhibitor, and accordingly, proceeded towards the evaluation of the HSP90 inhibition activity of the adducts (1-10).

### Example 4—HSP90 Inhibition

An in-vitro experiment was performed to assess the inhibitory effect of the synthesized adducts on the heat shock protein 90 (HSP90). Geldanamycin was employed as a standard, comparative HSP90 inhibitor. The results of the study are summarized in Table 2 below.

TABLE 2
HSP90 inhibitory activity of adducts 1-10; HSP90 inhibitory assays
were performed by reaction biology corporation, Malvern, PA

| | (HSP90a) |
|---|---|
| Compound ID: | IC₅₀ (nM) |
| 1 | — |
| 2 | — |
| 3 | 343 |
| 4 | 823 |
| 5 | — |
| 6 | >10000 |
| 7 | 60.1 |
| 8 | 63.6 |
| 9 | 9330 |
| 10 | 2840 |
| Geldanamycin | 20.9 |

It was a bit intriguing to observe that substitution at the amide NH (bearing the resorcinol fragment) had a galactic, substantial influence on the ability of the generated adducts to inhibit the chaperone protein. As such, a reversed pattern of increased HSP90 inhibitory activity was evidenced with N-alkylation/benzylation in this assay as compared to the results of the EZH2 inhibitory assay. Hybrid structures 1 and 2 were devoid of HSP90 inhibitory potential; however, methylation and ethylation at the amide nitrogen conferred HSP90 inhibitory potential to the scaffolds (3, 4, 7 and 8). Disappointingly, N-propyl and -benzyl substitutions (5, 6, 9 and 10) could not replicate the activity-conferring trends observed with the N-methyl and ethyl group, and it was conceived that the placement of the bulkier substituent was not tolerable at the amide NH (bearing the resorcinol fragment). Notably, the hybrid template featuring the amide bond connector (for the resorcinol fragment) at position 3′ benefited the most in the context of activation of the chemical architectures towards the HSP90 inhibitory activity. N-methyl and ethyl substitution at the amide NH of target scaffolds encompassing the resorcinol fragment at position 3′ culminated into two substantially potent HSP90 inhibitors, compounds 7 (IC₅₀ value=60.1 nM) and 8 (IC₅₀ value=63.6 nM). As observed in the EZH2 inhibitory evaluation study, the adducts replicated the trend of not being more potent than the standard commonly employed (geldanamycin, in HSP90 inhibitory assay and tazemetostat in EZH2 inhibitory assay). Unperturbed by the observed inferior profiles of the adducts in both the assays, pinpointing a balanced dual EZH2-HSP90 inhibitor 7 endowed with an IC₅₀ value of 6.29 nM (EZH2 inhibition) and 60.1 nM (HSP90 inhibition), respectively, was a matter of delight for our research group.

### Example 5—Itraq-Based Proteomics Analysis

Having identified the desired chemical probe 7, we assessed global protein expression using Itraq-based proteomics analysis to estimate whether 7 suppresses HSP90 (Pt3R cell lines). As shown in Tables 3-5, HSP family proteins, including HSPA1A, HSPA8, HSP90AA1, HSPB1, HSPH1 and HSPA4, were significantly increased in response to dual inhibitor 7 treatment. This observation is aligned with the results attained with geldanamycin treatment (a well-known HSP90 inhibitor) (Cheung, C. H. A.; Chen, H.-H.; Cheng, L.-T.; Lyu, K. W.; Kanwar, J. R.; Chang, J.-Y. TargetingHsp90 with small molecule inhibitors induces the over-expression of the anti-apoptotic molecule, survivin, in human A549, HONE-1 and HT-29 cancer cells. Mol. Cancer 2010, 9 (1), 1-11; Kuroyanagi, G.; Tokuda, H.; Fujita, K.; Kawabata, T.; Sakai, G.; Kim, W.; Hioki, T.; Tachi, J; Matsushima-Nishiwaki, R.; Otsuka, T. Upregulation of TGF-β-induced HSP27 by HSP90 inhibitors in osteoblasts. BMC Musculoskelet. Disord. 2022, 23 (1), 495) and confirms the HSP90-suppressive activity of compound 7 in GBM cells.

TABLE 3
Compound 7-influenced proteins grouped functionally by KEGG analysis.

| | Term in | | |
|---|---|---|---|
| Trend | KEGG_PATHWAY | Genes | P-Value |
| up-regulation | hsa04612: Antigen | HSPA8, HSP90AA1, | 7.88E−04 |
| | processing and presentation | HSPA4, HSPA1A | |
| up-regulation | hsa04141: Protein processing | HSPA8, HSP90AA1, | 7.69E−03 |
| | in endoplasmic reticulum | HSPH1, HSPA1A | |
| up-regulation | hsa04144: Endocytosis | ARF3, EHD1, HSPA8, | 2.01E−02 |
| | | HSPA1A | |
| up-regulation | hsa04915: Estrogen signaling | HSPA8, HSP90AA1, | 2.42E−02 |
| | pathway | HSPA1A | |
| up-regulation | hsa04390: Hippo signaling | SERPINE1, CTNNB1, | 5.25E−02 |
| | pathway | ACTB | |
| up-regulation | hsa05164: Influenza A | HSPA8, ACTB, | 6.75E−02 |
| | | HSPA1A | |
| up-regulation | hsa04510: Focal adhesion | ZYX, CTNNB1, ACTB | 9.04E−02 |
| down-regulation | hsa03010: Ribosome | RPS6, RPL35, RPL26, | 8.84E−03 |
| | | RPS10 | |
| down-regulation | hsa04110: Cell cycle | MCM7, MCM3, | 5.89E−02 |
| | | YWHAG | |
| down-regulation | hsa03040: Spliceosome | HNRNPA3, PCBP1, | 6.66E−02 |
| | | SRSF7 | |

TABLE 4
Proteins increased by Compound 7 (2 mM)
treatment analyzed by iTRAC assay.

| Accession | Gene name | Score | M059/DMSO |
|---|---|---|---|
| P35222 | CTNNB1 | 137.20 | 1.550 |
| Q9H4M9 | EHD1 | 59.69 | 1.555 |
| P34932 | HSPA4 | 312.76 | 1.561 |
| O00487 | PSMD14 | 57.97 | 1.579 |
| O15460 | P4HA2 | 105.19 | 1.588 |
| P36543 | ATP6V1E1 | 41.77 | 1.599 |
| Q92598 | HSPH1 | 123.02 | 1.642 |
| Q9UII2 | ATP5IF1 | 45.22 | 1.646 |
| P60709 | ACTB | 1504.25 | 1.657 |
| P04792 | HSPB1 | 264.25 | 1.661 |
| P60903 | S100A10 | 243.10 | 1.664 |
| P30533 | LRPAP1 | 80.20 | 1.667 |
| O14908 | GIPC1 | 27.10 | 1.699 |
| P61604 | HSPE1 | 133.46 | 1.710 |
| Q9NTK5 | OLA1 | 37.59 | 1.745 |
| P24534 | EEF1B2 | 75.42 | 1.792 |
| O43242 | PSMD3 | 36.87 | 1.801 |
| Q15942 | ZYX | 24.18 | 1.903 |
| P19367 | HK1 | 88.39 | 1.921 |
| P61204 | ARF3 | 41.00 | 1.944 |
| P07900 | HSP90AA1 | 958.86 | 1.958 |
| Q12792 | TWF1 | 39.76 | 1.995 |
| P11142 | HSPA8 | 959.46 | 2.096 |
| Q56VL3 | OCIAD2 | 41.53 | 2.150 |
| P50454 | SERPINH1 | 113.69 | 2.186 |
| O00299 | CLIC1 | 74.34 | 2.598 |
| P0DMV8 | HSPA1A | 469.78 | 2.843 |
| P15531 | NME1 | 188.40 | 3.299 |
| P05121 | SERPINE1 | 50.02 | 4.203 |

TABLE 5
Proteins decreased by Compound 7 (2 mM)
treatment analyzed by iTRAC assay.

| Accession | Gene name | Score | M059/DMSO |
|---|---|---|---|
| Q71DI3 | HIST2H3A | 93.71 | 0.240 |
| Q96KK5 | HIST1H2AH | 157.12 | 0.245 |
| P16403 | H1-2 | 552.01 | 0.334 |
| P51991 | HNRNPA3 | 121.56 | 0.377 |
| P08758 | ANXA5 | 30.37 | 0.406 |
| Q9ULV4 | CORO1C | 53.84 | 0.410 |
| O60814 | H2BC12 | 211.06 | 0.419 |
| P06899 | H2BC11 | 220.59 | 0.423 |
| P21589 | NT5E | 71.63 | 0.446 |
| P61981 | YWHAG | 112.04 | 0.447 |
| O00148 | DDX39A | 156.38 | 0.515 |
| P62805 | H4C1 | 598.09 | 0.556 |
| P68371 | TUBB4B | 763.06 | 0.564 |
| O43169 | CYB5B | 36.86 | 0.568 |
| O15347 | HMGB3 | 22.25 | 0.573 |
| P26583 | HMGB2 | 128.00 | 0.575 |
| Q68CZ2 | TNS3 | 52.27 | 0.582 |
| P33993 | MCM7 | 50.38 | 0.586 |
| Q15185 | PTGES3 | 125.85 | 0.589 |
| P55084 | HADHB | 35.40 | 0.591 |
| P26196 | DDX6 | 92.60 | 0.596 |
| P0C0S5 | H2AZ1 | 180.00 | 0.603 |
| P49321 | NASP | 150.44 | 0.604 |
| Q16629 | SRSF7 | 31.45 | 0.605 |
| P46783 | RPS10 | 88.48 | 0.608 |
| Q92804 | TAF15 | 55.54 | 0.609 |
| P61254 | RPL26 | 82.88 | 0.619 |
| Q13347 | EIF3I | 65.65 | 0.624 |
| P63244 | RACK1 | 121.61 | 0.627 |
| P62879 | GNB2 | 118.48 | 0.628 |
| Q15365 | PCBP1 | 80.97 | 0.628 |
| Q9UQ80 | PA2G4 | 235.25 | 0.628 |
| P42766 | RPL35 | 20.64 | 0.629 |
| P35237 | SERPINB6 | 27.22 | 0.632 |
| Q8NBS9 | TXNDC5 | 22.91 | 0.635 |
| P25205 | MCM3 | 44.10 | 0.635 |
| Q99584 | S100A13 | 44.16 | 0.638 |
| P62753 | RPS6 | 41.92 | 0.641 |
| Q12906 | ILF3 | 103.00 | 0.645 |

### Example 6—Structure-Based Molecular Docking

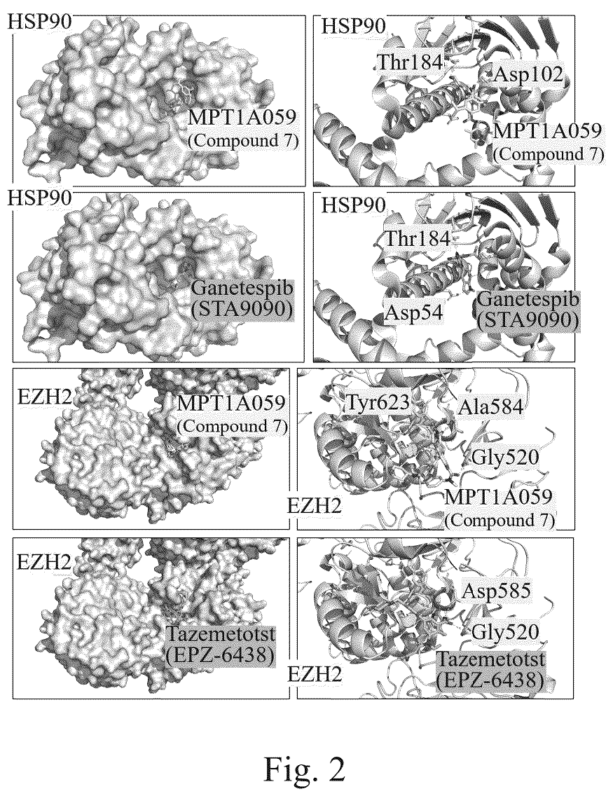

Structure-based molecular docking was performed to rationalize the experimental results of the enzymatic assays (FIG. 2). The docking process was carried out between protein and compounds with a population size of 1500, generations of 150, and solutions of 10 through iGEMDOCK version 2.1. Protein structures of HSP90 and EZH2 were obtained from Protein Data Bank (8AGI and 4W2R). Notably, compound 7 was found to be associated with the ATP-binding pocket of HSP90, which is a target of a well-known HSP90 inhibitor, ganetespib (STA9090) [51]. In addition, similar to an EZH2 inhibitor, tazemetostat, compound 7 was observed to be associated with a site which is responsible to mediate the methyl transfer reaction of EZH2 [52]. These results indicated that compound 7 has high specificity targeting ability to HSP90 and EZH2.

### Example 7—In-Vitro Cytotoxicity Studies

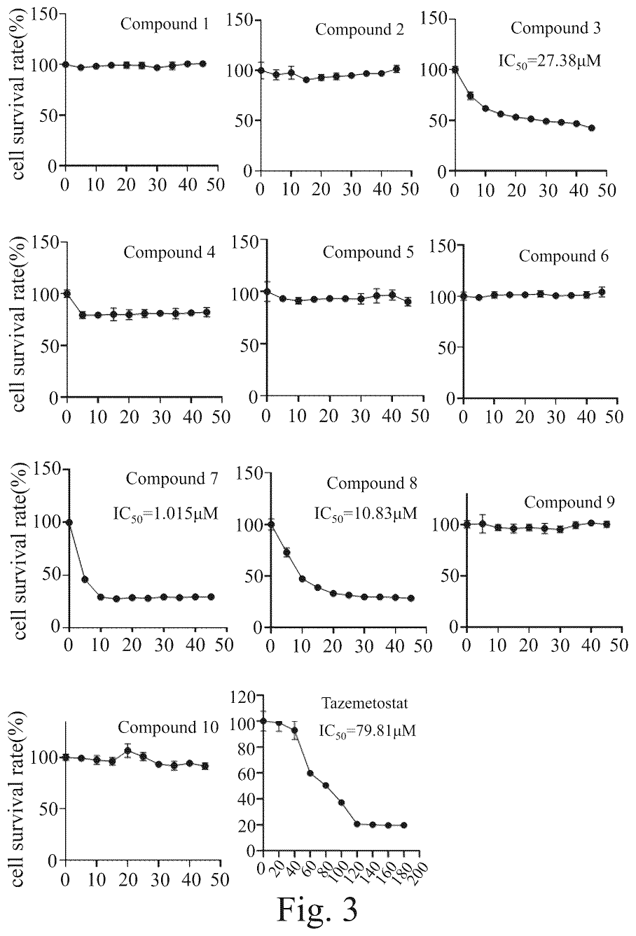

In-vitro cytotoxicity studies were carried out to validate whether the hypothesis of extracting anti-GBM effects via balanced dual modulation of EZH2 and HSP90 corroborates with the experimental results (Table 6, FIG. 3). Thus, the adducts (1-10) were evaluated for their ability to inhibit the cell viability of TMZ-resistant cells (Pt3-R). Delightfully, the most potent and balanced EZH2-HSP90 dual inhibitor 7 demonstrated significant cell proliferation inhibitory potential against Pt3-R cell lines with an IC₅₀ value of 1.015 μM. A correlation among the results of Tables 1, 2 and 6 indicates that the adducts (1, 2, 4-6, 9 and 10) as well as tazemetostat solely acting as EZH2 inhibitors were unable to exert cell growth inhibitory effects, and that modulation of both the targets (EZH2 and HSP90) was mandatory for the anti-GBM efficacy. Notably, compounds 3, 7 and 8, which can be categorised as dual inhibitors, were the only ones to exert cell viability inhibitory potential. Amongst them, compound 7 [the most balanced and potent dual inhibitor of the targets (EZH2-HSP90)] was the most effective against the Pt3R cell lines. Given the results, it is perspicuous, evident that the magnificent enzyme inhibitory profile of compound 7 translated into cytotoxicity against the TMZ-resistant cells (Pt3-R) GBM cell lines. In a nutshell, it is deduced that cell growth inhibitory effects of compound 7 stem from its ability to modulate EZH2 and HSP90 chaperone in a balanced manner.

TABLE 6
Cell growth inhibitory effects of Compounds 1-10

| Drug list | |
|---|---|
| (Compound No.) | IC50 (μM) |
| 1 | — |
| 2 | — |
| 3 | 27.38 |
| 4 | |
| 5 | — |
| 6 | — |
| 7 | 1.015 |
| 8 | 10.83 |
| 9 | — |
| 10 | — |
| Tazemetostat | 79.81 |

—: undetectable

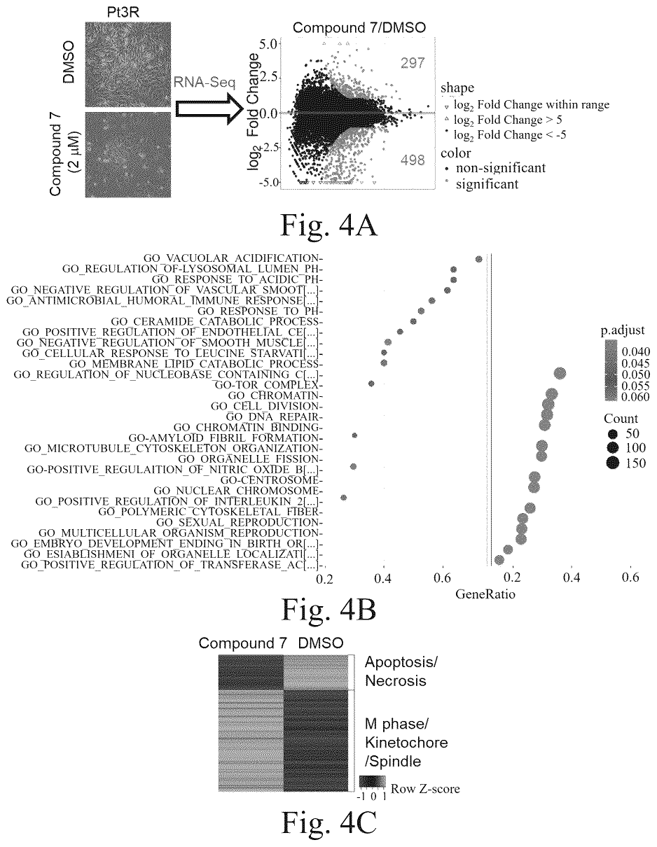

FIG. 4A shows that after treatment for 48 h, RNA extracts were collected and subjected to RNA-seq. Left, Cell morphology; Right, genes significantly influenced by compound 7. FIG. 4B shows GSEA functionally grouped genes. FIG. 4C shows that genes involved in apoptosis/necrosis and M phase/Kinetochore/Spindle were clustered in the heatmap.

### Example 8—Dual Inhibitor 7 Suppressed Kinetochore- and DNA Repair-Related Gene Expression

To gain insights regarding the ability of dual inhibitor 7 to suppress the viability of TMZ-resistant GBM cells, RNA-seq to analyze gene expression profiles was performed. As shown in FIG. 4A, treatment of Pt3R with 2 μM of 7 for 48 h significantly inhibited cell proliferation. Also, RNA-seq revealed that compound 7 increased and decreased 297 and 498 gene expressions, respectively. Furthermore, the outcome of Gene Set Enrichment Analysis (GSEA) of compound 7 indicated that cell division and DNA repair were highly suppressed (FIG. 4B). Notably, treatment with compound 7 increased apoptosis/necrosis-related gene expression, whereas it decreased M phase/kinetochore/spindle-related gene expression (FIG. 4C).

### Example 9—Decreased CENPs Protein Expression

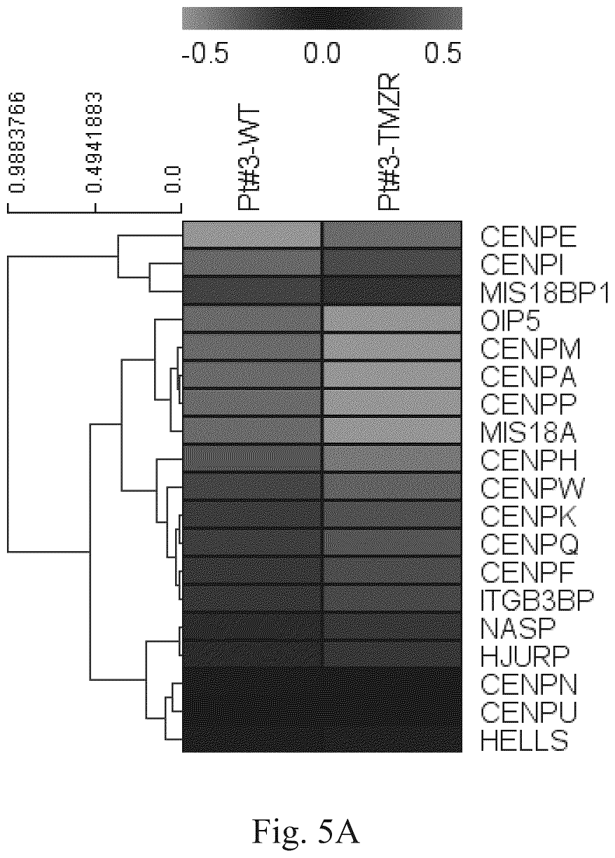

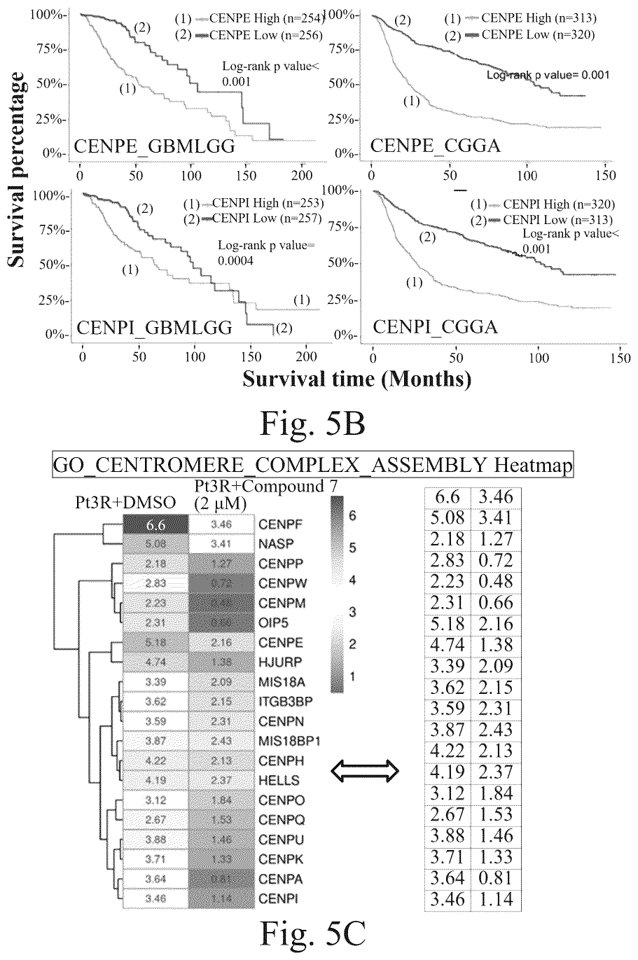

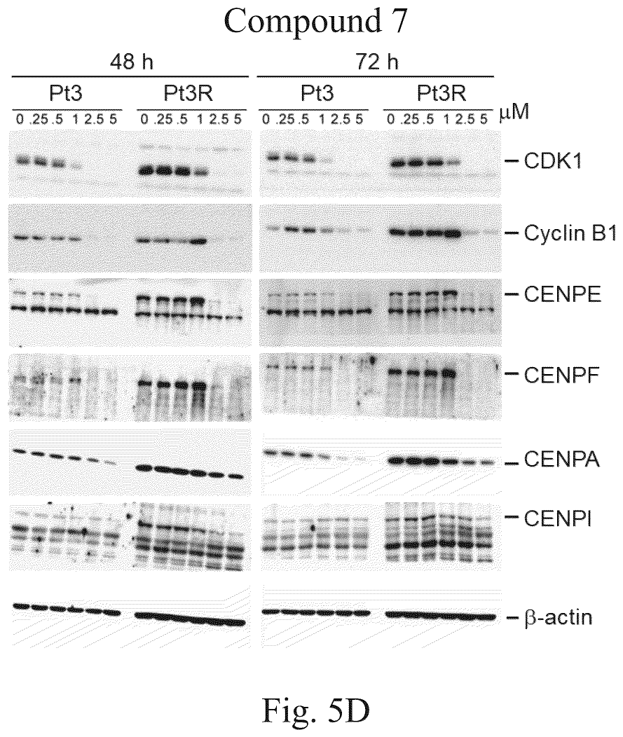

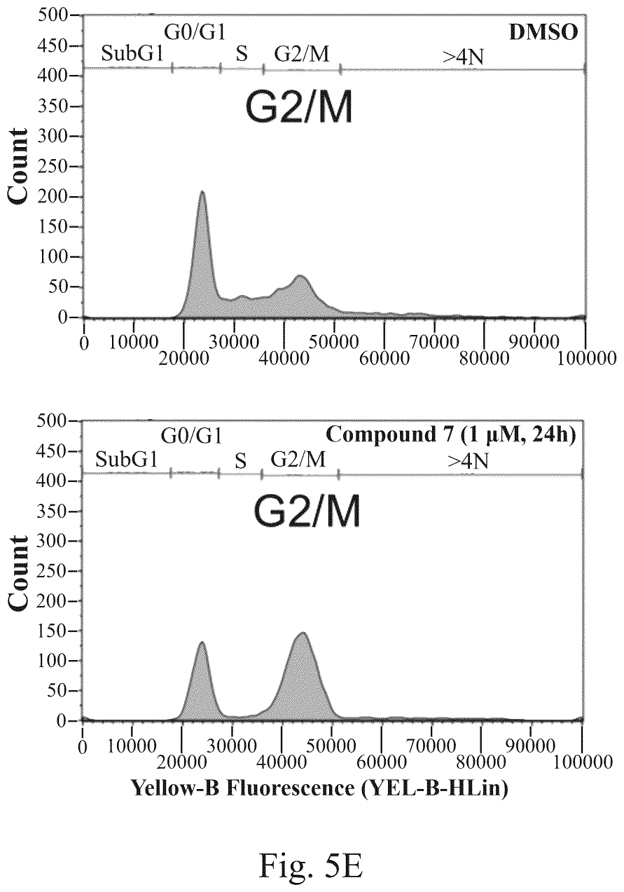

Furthermore, the impact of compound 7 treatment on centromere proteins (CENPs) was assessed. As such, CENPs are the main constituent proteins of the kinetochore, which are essential for cell division. CENP expression is elevated in GBM tissues and correlated with unfavourable overall survival in glioma patients [53]. Before evaluating the effect of dual inhibitor 7, a study to assess the expression levels of CENPs was carried out and the upregulated expression of CENPE and CENPI was evidenced in TMZ-resistant GBM cells (Pt3R) compared to patient-derived GBM cells, Pt3 (FIG. 5A). It was also observed that CENPE and CENPI were significantly correlated with poor prognosis of GBM patients (FIG. 5B), suggesting that CENPs are important for GBM progression. Delightfully, further investigation results revealed that centromere proteins (CENPs) including CENPF, CENPE, CENPA and CENPI, which are important to regulate kinetochore assembly and mitosis, were decreased by compound 7 treatment (FIG. 5C). Western blot analysis was also performed and the outcome indicated that hybrid scaffold 7 also downregulated the expression of CDK1 and cyclin B1, both of which are required to progress M phase (FIG. 5D). Additionally, CENPs protein expression was also decreased by 7 in Pt3R cells (FIG. 5D). Also, dual inhibitor 7 induced cell cycle arrest at the M phase (FIG. 5E).

FIG. 5A shows the Compound 7-suppressed CENP family. FIG. 5B shows gene expression of the CENP family in Pt3 and TMZ-resistant Pt3R cells. FIG. 5C shows that prognostic value was analyzed using the GlioVis website. FIG. 5D shows that Western blotting was used to confirm that compound 7 decreased CENP expression. FIG. 5E shows cell cycle analysis: after treatment for 24 h, cells were stained with propidium iodide (PI) and subjected to flow cytometry analysis.

### Example 9—Downregulated the DNA Repair-Related Gene Expression

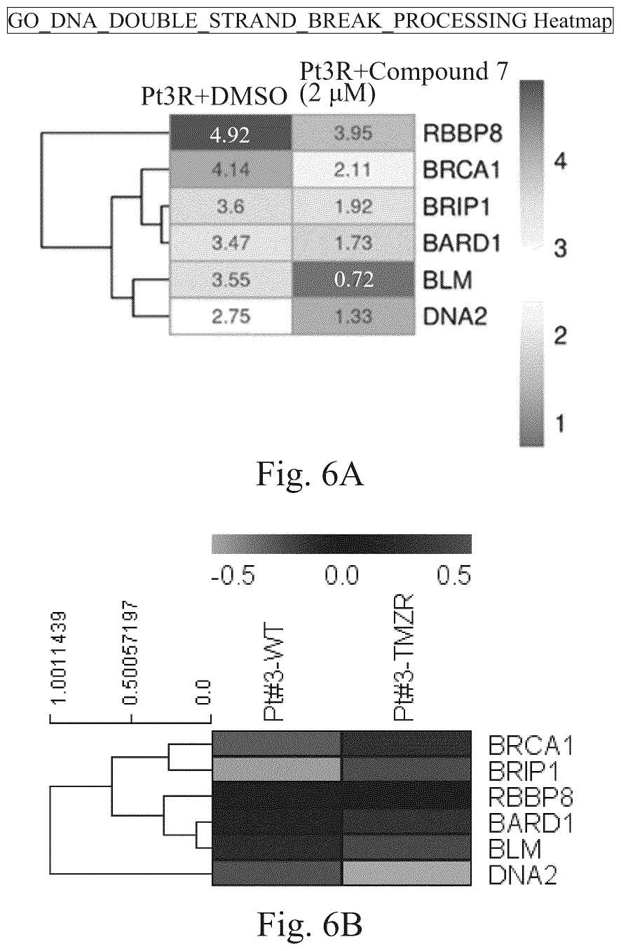

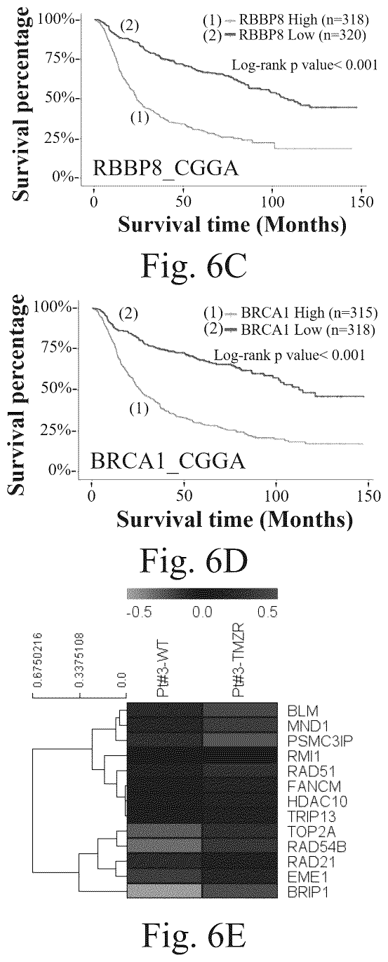

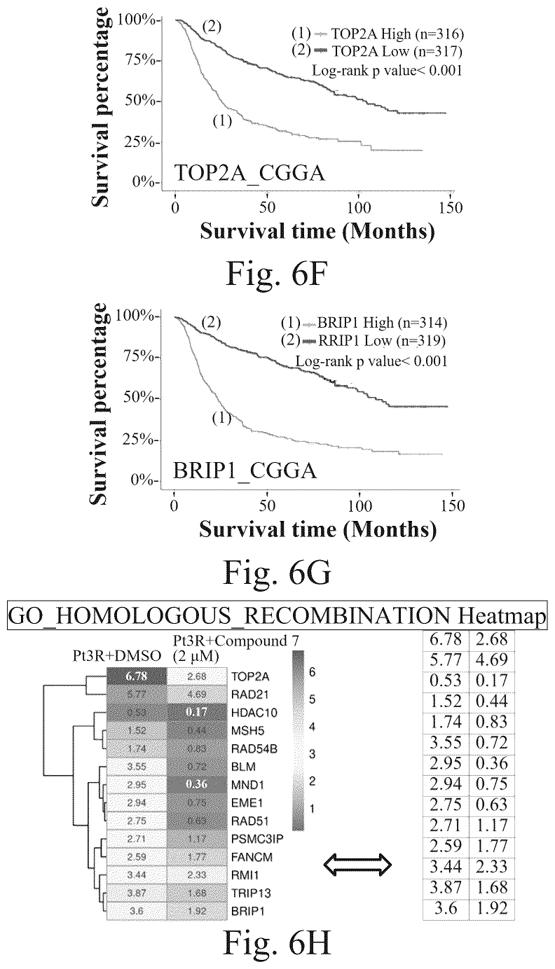

In addition to cell division, DNA repair capacity was highly suppressed by compound 7. In particular, expression of genes involved in processing DNA double-strand break and in homologous recombination was decreased by 7 (FIG. 6A). Further explorations conducted to establish the expression profile of genes in TMZ-resistant Pt3R cells revealed that RB Binding Protein (RBBP8) and BRCA1 expression were increased in TMZ-resistant Pt3R cells (FIG. 6B) as per the relative comparison of their expression levels in Pt3 cells. Also, these 2 genes (RBPP8 and BRCA1) were significantly correlated with poor prognosis of GBM patients (FIG. 6C-D). Notably, both were suppressed by treatment with compound 7 (FIG. 6A). Amongst the genes regulating homologous recombination, DNA topoisomerase II (TOP2A), DNA repair and recombination protein RAD54B, RAD21, crossover junction endonuclease EME1 and BRIP1 were found to be increased in TMZ-resistant Pt3R cells as compared with Pt3 cells (FIG. 6E). Particularly, TOP2A and BRCA1 interacting helicase (BRIP) 1 significantly correlated with poor prognosis (FIG. 6F-G). Delightfully, the genes mentioned above which regulate homologous recombination were suppressed by compound 7 (FIG. 6H). Taken together, these findings highlighted the potential of compound 7 to downregulate the DNA repair-related gene expression which is assumed to be responsible for the anti-GBM efficacy of compound 7.

FIG. 6A shows the effect of compound 7 on gene expression involved in processing DNA double-strand break. FIG. 6B shows the comparison in gene expression between Pt3 and Pt3R cells. FIGS. 6C-6D show the prognostic value of RBBP8 and BRCA1 in GBM patients. FIG. 6E shows the comparison in gene expression between Pt3 and Pt3R cells. FIGS. 6F-6G show the prognostic value of TOP2A and BRIP1 in GBM patients. FIG. 6H shows the effect of compound 7 on gene expression involved in homologous recombination.

### Example 10—Dual Inhibitor Increased ROS Accumulation by Disrupting Redox Homeostasis in Mitochondria

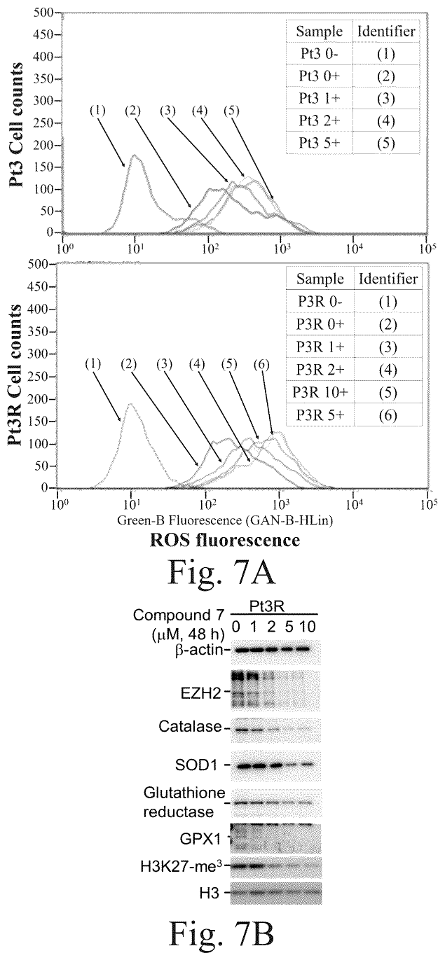

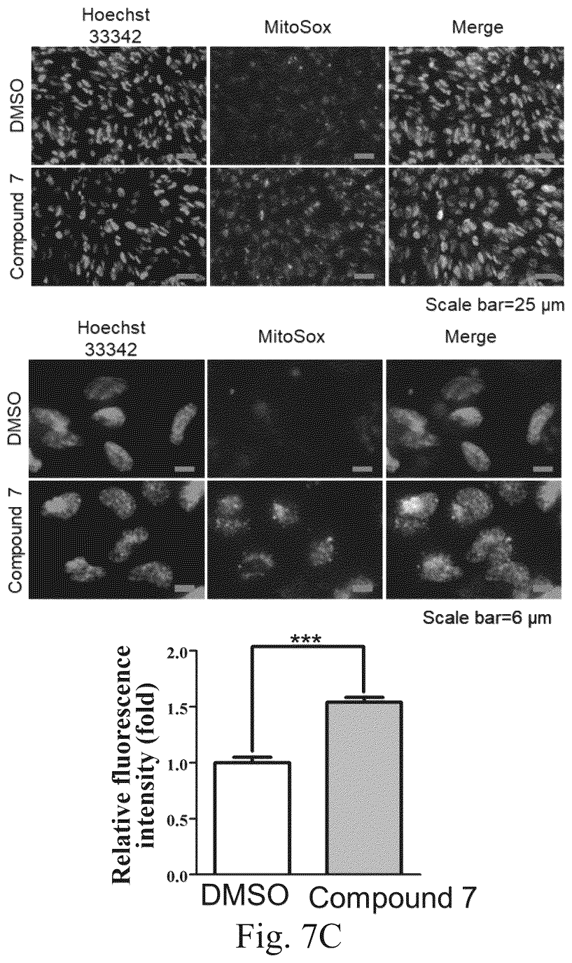

Literature precedents revealed that TMZ-resistant GBM cells can survive in the presence of TMZ through enhanced reactive oxygen species (ROS)-cleaning capacity [54-57]. Thus, the ability of compound 7 to disturb redox homeostasis in TMZ-resistant cells and attain amplified therapeutic efficacy for GBM was evaluated. In both Pt3 and Pt3R cells, treatment with compound 7 for 48 h increased the ROS level (FIG. 7A). Moreover, proteins which are involved in cleaning ROS, including catalase, superoxide dismutase (SOD) 1, glutathione reductase and glutathione peroxidase (GPX) 1, were also decreased by compound 7 (FIG. 7B). In parallel, EZH2 expression and EZH2-modulated H3K27 trimethylation were also inhibited by compound 7, indicating its inhibitory effect on EZH2 (FIG. 7B). These results indicated that compound 7 suppressed the ROS catabolism pathway, causing the death of TMZ-resistant GBM cells. Moreover, it was also observed that compound 7 increased mitochondria-derived ROS. To assess the mitochondria-derived ROS increasing potential of compound 7, fluorescent MitoSOX labelled mitochondrial ROS was used, and it was found that compound 7 remarkably enhanced the red signal representing ROS produced from mitochondria (FIG. 7C)

FIG. 7A shows that after treatment for 48 h, cells were stained with the DHR reagent, and the fluorescent signal was estimated by flow cytometry. Treatment concentration of MPT1A059 was indicated. FIG. 7B shows that protein lysates were subjected to Western blotting using the indicated antibody. FIG. 7C shows that after staining with MitoSOX (red) and Hoechst 33342 (blue), cellular signals were photographed and quantified.

### Example 11—Dual Inhibitor Exhibited Magnificent Potential to Inhibit the Growth of TMZ-Resistant GBM In Vivo

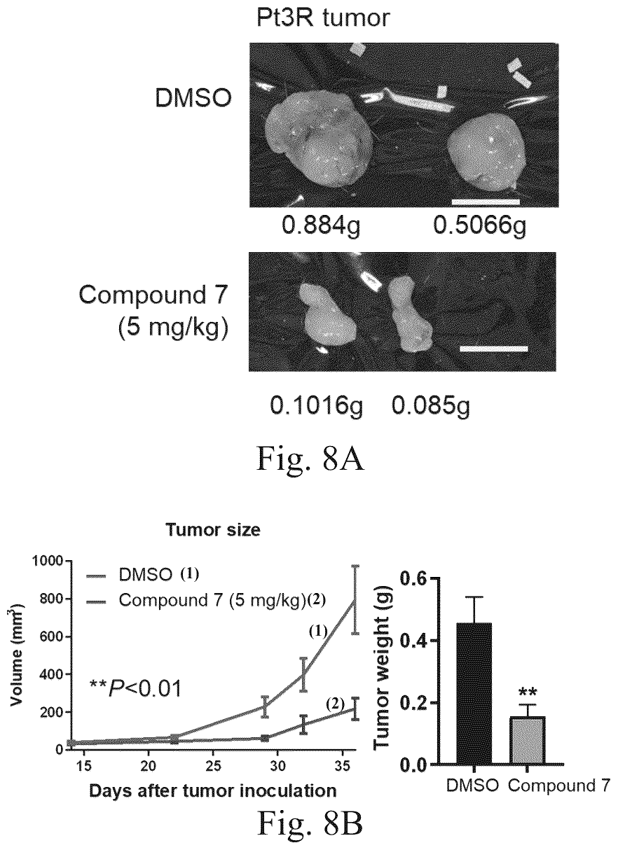

In-vivo anti-GBM efficacy of compound 7 was also evaluated in this study. For the in-vivo study, experimental NOD.CB17-Prkdc^{scid}/NCrCrl mice (8-week-old) were used and Pt3R cells (1×10⁶) in 50 ml DMEM were injected into the backs of mice. After 10 days, the tumor was detectable. Mice were injected with DMSO (the control group) or compound 7 (5 mg/kg) twice weekly. As shown in FIG. 8, tumors in compound 7-treated mice exhibited significantly smaller sizes and weights compared to those in DMSO-treated mice, indicating the therapeutic efficacy of compound 7 in vivo (FIG. 8A). Moreover, results presented in FIG. 8B demonstrated the tumor growth suppression ability of compound 7. In a nutshell, the outcome of the in-vivo study ascertains that the remarkable in vitro anti-GBM activity profile of dual inhibitor 7 was translated to the in-vivo potential.

FIG. 8A shows the representative photograph of Pt3R tumors. FIG. 8B shows growth curve of tumor and tumor weight.

Numerous precedents coupled with our investigation to assess the expression level of EZH2 tissues advocated for its overexpression in GBM tissues. Also, the high expression of EZH2 significantly correlated with the shorter survival time in patients with GBM, affirming its role as an important oncogene in GBM pathogenesis. Intriguingly, the only FDA-approved EZH2 inhibitor, Tazemetostat, did not exhibit anti-GBM efficacy, and the idea of stitching another antitumor pharmacophore to the core structure of tazemetostat was conceived as a prudent strategy to activate its chemical architecture to exert anti-GBM effects. Notably, this conceivement or realization was predominantly attributed to the knowledge gained from our previous dual inhibitor fabrication campaigns. The candidature of the HSP90 chaperone protein inhibitors was deemed suitable for the tetheration to the structural template of Tazemetostat, in light of numerous reports validating the efficacy of HSP90 inhibitors in GBM coupled with our preliminary investigation results confirming the possible attainment of remarkable anti-GBM efficacy through a combination of EZH2 and HSP90 inhibitors. Thus, to extract pronounced anti-GBM effects from balanced modulation of EZH2 and HSP90, hybrid templates comprising structural commonalities of EZH2 and HSP90 inhibitors were constructed via multistep synthetic routes. Encouragingly, a strikingly balanced dual inhibitor 7 was identified through the in-vitro enzymatic assays, and the impact of dual inhibition was evidenced in the cytotoxicity studies. Hybrid template 7 displayed substantial cell growth inhibitory activity against Pt3R that was presumably attributed to its dual EZH2-HSP90 inhibitory potential. A further exhaustive exploration of chemical probe 7 ascertained its ability to i) suppress kinetochore- and DNA repair-related gene expression, ii) increase ROS accumulation through disrupting redox homeostasis in mitochondria, and iii) inhibit the growth of TMZ-resistant GBM in vivo. Overall, the study has resulted in identifying a tractable dual inhibitor that encompasses the requisite features of an emerging therapeutic for treatment-resistant brain tumors.

A person of ordinary skill in the art of the subject disclosure should understand that variations and modifications may be made to the teaching and the disclosure of the subject disclosure without departing from the spirit and scope of the subject application. Based on the contents above, the subject application intends to cover any variations and modifications thereof with the proviso that the variations or modifications fall within the scope as defined in the appended claims or their equivalents.

What is claimed is:

1. A compound of Formula (I), 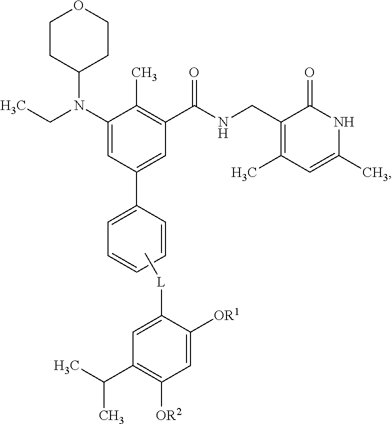
or a pharmaceutically acceptable salt,
hydrate, solvate or prodrug thereof,
wherein L is a linker selected from the group consisting of -A-NR—C(═Y)—B— and -A-C(═Y)—NR—B—;
A and B are independently selected from the group consisting of a direct bond, C₁-C₃ alkyl and C₁-C₃ alkoxy;
R is selected from the group consisting of H, linear or branched C₁-C₆ alkyl, linear or branched C₁-C₆ alkoxy, C₆-C₁₀ aryl and C₅-C₁₀ heteroaryl;
Y is selected from the group consisting of O, S and N; and
R¹ and R² are independently selected from H, halo, C₁-C₆ alkyl and C₁₆-C₂₀ (poly)unsaturated fatty acids;
wherein the alkyl and alkoxyl can be optionally substituted by halogen, hydroxy, amino or C₆-C₁₀ aryl or C₅-C₁₀ heteroaryl; and
wherein the aryl and heteroaryl can be optionally substituted by halogen, hydroxy, amino, C₁-C₃ alkyl or C₁-C₃ alkoxy.

2. The compound of claim 1, or a pharmaceutically acceptable salt, hydrate, solvate or prodrug thereof, wherein L is -A-NR—C(═Y)—B— and -A-C(═Y)—NR—B—, wherein Y is O; A is a direct bond, B is a direct bond and R is H and R¹ and R² are each independently H, halo, C₁-C₆ alkyl and oleic acid and linolenic acid (such as α- or γ-linolenic acid).

3. The compound of claim 1, or a pharmaceutically acceptable salt, hydrate, solvate or prodrug thereof, wherein L is -A-NR—C(═Y)—B— and -A-C(═Y)—NR—B—, wherein Y is O; A is a direct bond, B is a direct bond and R is linear or branched C₁-C₆ alkyl and R¹ and R² are each independently H, oleic acid and linolenic acid (such as α- or γ-linolenic acid).

4. The compound of claim 1, or a pharmaceutically acceptable salt, hydrate, solvate or prodrug thereof, wherein L is -A-NR—C(═Y)—B— and -A-C(═Y)—NR—B—, wherein Y is O; A is a direct bond, B is a direct bond and R is C₆-C₁₀ aryl (such as phenyl or benzyl) and R¹ and R² are each independently H, oleic acid and linolenic acid (such as α- or γ-linolenic acid).

5. The compound of claim 1, or a pharmaceutically acceptable salt, hydrate, solvate or prodrug thereof, wherein L is -A-NR—C(═Y)—B— and -A-C(═Y)—NR—B—, wherein Y is O; A is a direct bond, B is a direct bond and R is linear or branched C₁-C₆ alkyl optionally substituted by C₆-C₁₀ aryl; and R¹ and R² are each independently H, oleic acid and linolenic acid (such as α- or γ-linolenic acid).

6. The compound of claim 1, or a pharmaceutically acceptable salt, hydrate, solvate or prodrug thereof, wherein Formula (I) is Formula (Ia) or (Ib), 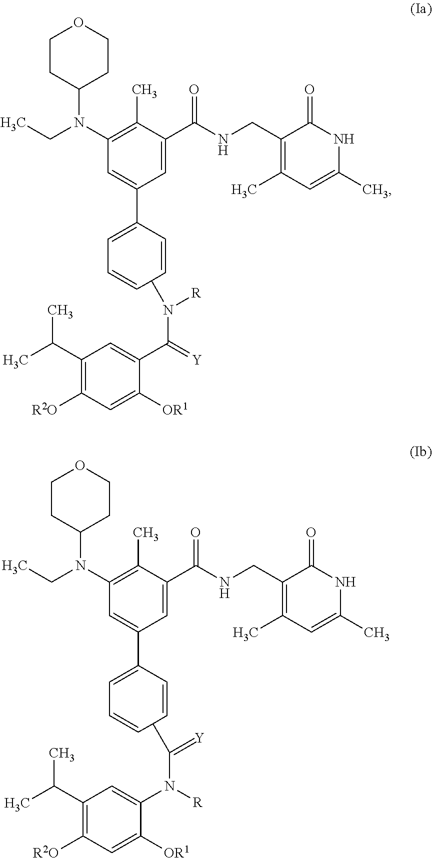
wherein:
Y is O or S;
R is H, linear or branched C₁-C₄ alkyl, phenyl or benzyl; and
R¹ and R² are independently selected from H, halo, C₁-C₆ alkyl and C₁₆-C₂₀ (poly)unsaturated fatty acids;
wherein the linear or branched C₁-C₄ alkyl is optionally substituted by halogen, hydroxy or amino; and
wherein the phenyl or benzyl is optionally substituted by C₁-C₂ alkyl, halogen, hydroxy or amino.

7. The compound of claim 1, or a pharmaceutically acceptable salt, hydrate, solvate or prodrug thereof, wherein Formula (I) is Formula (Ia-p) or (Ib-p), 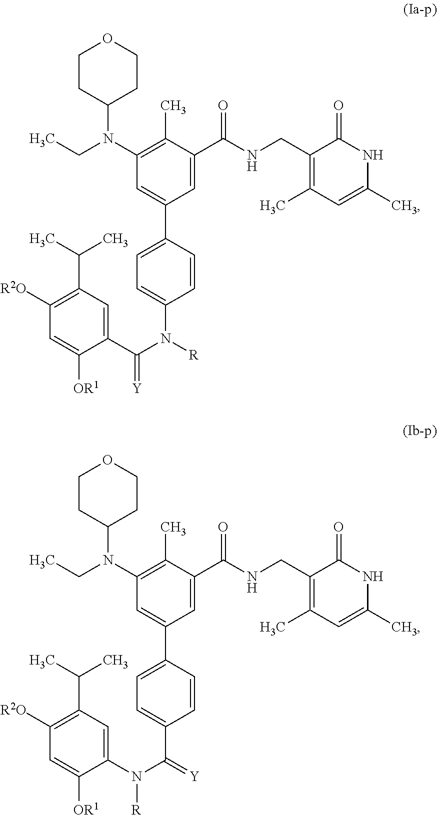
wherein:
Y is O or S;
R is H, linear or branched C₁-C₄ alkyl, phenyl or benzyl; and
R¹ and R² are independently selected from H, halo, C₁-C₆ alkyl and C₁₆-C₂₀ (poly)unsaturated fatty acids;
wherein the linear or branched C₁-C₄ alkyl is optionally substituted by halogen, hydroxy or amino; and
wherein the phenyl or benzyl is optionally substituted by C₁-C₂ alkyl, halogen, hydroxy or amino.

8. The compound of claim 1, or a pharmaceutically acceptable salt, hydrate, solvate or prodrug thereof, wherein Formula (I) is Formula (Ia-m) or (Ib-m), 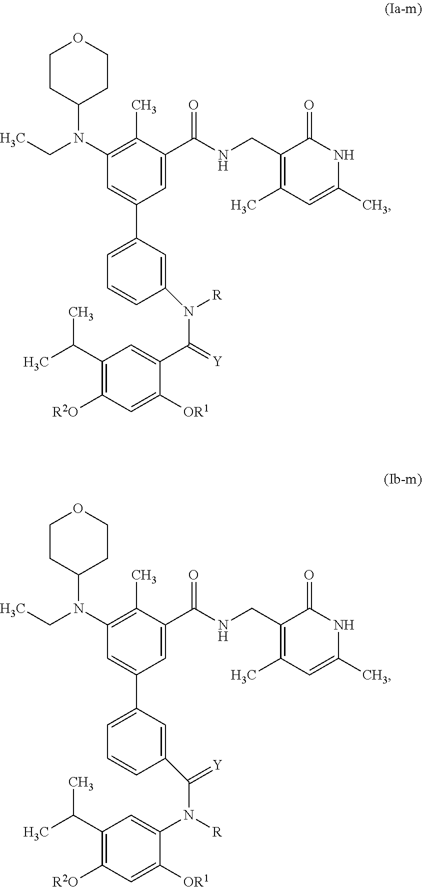
wherein:
Y is O or S;
R is H, linear or branched C₁-C₄ alkyl, phenyl or benzyl; and
R¹ and R² are independently selected from H, halo, C₁-C₆ alkyl and C₁₆-C₂₀ (poly)unsaturated fatty acids;
wherein the linear or branched C₁-C₄ alkyl is optionally substituted by halogen, hydroxy or amino; and
wherein the phenyl or benzyl is optionally substituted by C₁-C₂ alkyl, halogen, hydroxy or amino.

9. The compound of claim 6, or a pharmaceutically acceptable salt, hydrate, solvate or prodrug thereof, wherein Formula (Ia) is Formula (Ia-p) as described in claim 3 or Formula (Ia-m) as described in claim 4.

10. The compound of claim 1, which is selected from: 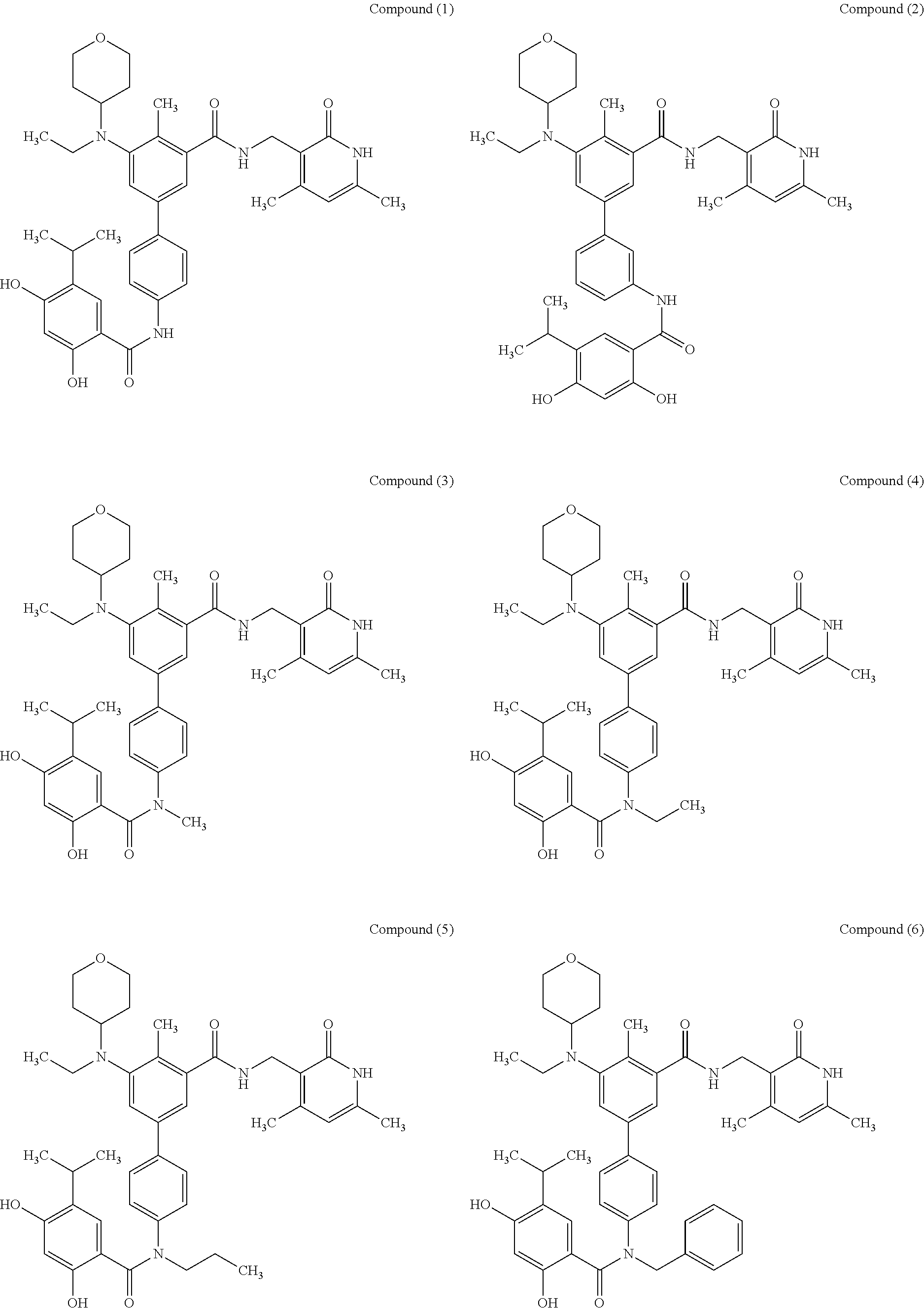 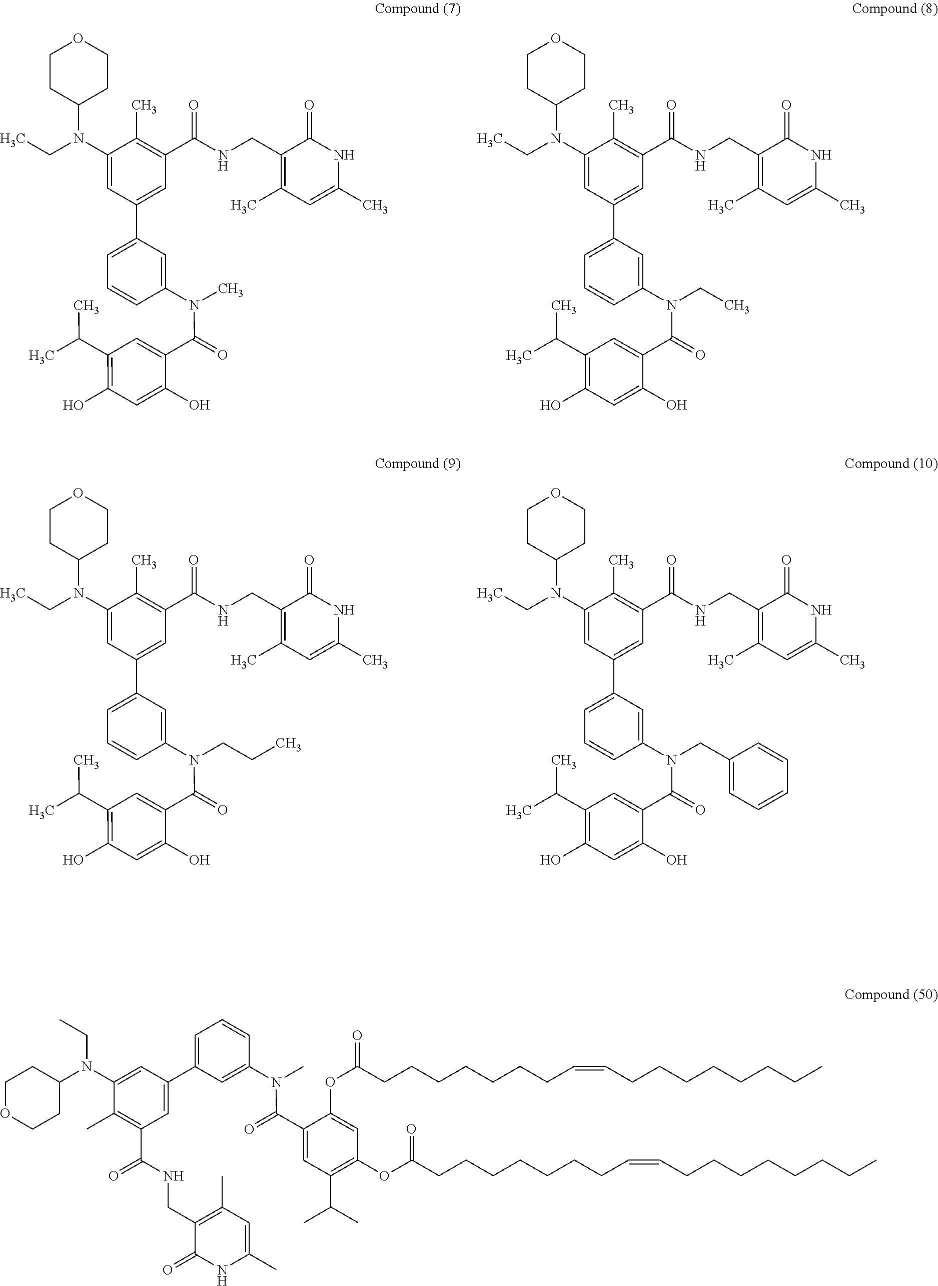 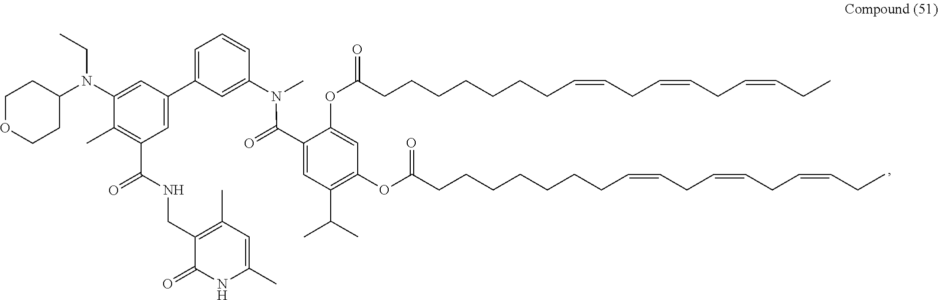 [SMILES: CC/C=C\C/C=C\C/C=C\CCCCCCCC(=O)Oc1cc(OC(=O)CCCCCCC/C=C\C/C=C\C/C=C\CC)c(C(C)C)cc1C(=O)N(C)c1cccc(-c2cc(C(=O)NCc3c(C)cc(C)[nH]c3=O)c(C)c(N(CC)C3CCOCC3)c2)c1]
or a pharmaceutically acceptable salt, hydrate, solvate or pro rug thereof.

11. The compound of claim 1, which is the compound having the formula: 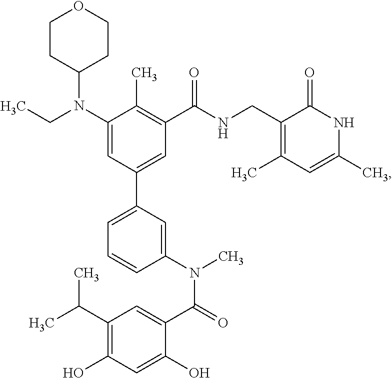 [SMILES: CCN(c1cc(-c2cccc(N(C)C(=O)c3cc(C(C)C)c(O)cc3O)c2)cc(C(=O)NCc2c(C)cc(C)[nH]c2=O)c1C)C1CCOCC1]
or a pharmaceutically acceptable salt, hydrate, solvate or prodrug thereof.

12. A pharmaceutical composition or pharmaceutical formulation comprising the compound or a pharmaceutically acceptable salt, hydrate, solvate or prodrug thereof according to claim 1.

13. A method of treating a disease or disorder mediated by EZH2, HSP90 or both in a patient, comprising administering the compound or a pharmaceutically acceptable salt, hydrate, solvate or prodrug thereof according to claim to a subject in need thereof.

14. The method according to claim 13, wherein the disease or disorder is a brain cancer.

15. The method of claim 14, wherein the brain cancer is a primary brain tumor or a metastatic brain cancer.

16. The method of claim 14, wherein the brain cancer is glioma or meningioma.

17. The method of claim 14, wherein the brain cancer is glioblastoma (GBM).
